(19) United States
(12) Patent Application Publication
(10) Pub. No.: US 2017/0203009 A1
(43) Pub. Date: Jul. 20, 2017

(54) Biomimetic Scaffold for Regenerative Dentistry

(71) Applicant: Temple University-Of The Commonwealth System of Higher Education, Philadelphia, PA (US)

(72) Inventors: Maobin Yang, Wynnewood, PA (US); Peter I. Lelkes, Cherry Hill, NJ (US); Riddhi A. Gangolli, Philadelphia, PA (US); Jonathan A. Gerstenhaber, Philadelphia, PA (US); Sean M. Devlin, Philadelphia, PA (US)

(73) Assignee: Temple University-Of The Commonwealth System of Higher Education, Philadelphia, PA (US)

(21) Appl. No.: 15/326,189

(22) PCT Filed: Jul. 14, 2015
(86) PCT No.: PCT/US15/40391

§ 371 Date: Jan. 13, 2017

Related U.S. Application Data
(60) Provisional application No. 62/024,180, filed on Jul. 14, 2014.

(51) Int. Cl. A61L 27/58; A61L 27/56; A61L 27/18

(52) U.S. Cl. CPC A61L 27/58; A61L 27/18; A61L 27/56; A61L 2430/12

(57) ABSTRACT

The present invention relates to biomimetic scaffolds, methods for making the same, and methods for using the same. The scaffolds comprise a plurality of graded or tapered microchannels that provide spatial control for cell penetration. The scaffolds are useful for regenerating missing interface tissue between two adjacent tissues, or regeneration and integration of two adjacent tissues directly.

### CROSS-REFERENCE TO RELATED APPLICATIONS

This application claims priority to U.S. Provisional Patent Application No. 62/024,180 filed Jul. 14, 2014, the contents of which are incorporated by reference herein in its entirety.

### BACKGROUND OF THE INVENTION

Periodontitis is a chronic disease causing progressive destruction of the tooth-supporting tissues and eventually leads to tooth loss. According to CDC data from 2009 and 2010, about 47.2% of adults (64.7 million) in the United States had periodontitis, and about 38.5% of adults had moderate or severe forms of periodontitis which causes progressive destruction of the periodontal ligament (PDL) and alveolar bone. In adults ages 65 and older, the prevalence was 70.1%, which has surpassed diabetes. Unless addressed proactively or treated appropriately, periodontitis can result in tooth loss. In addition, it has been well proved that periodontitis is associated with systemic diseases such as heart disease and diabetes.

Current treatment modalities of periodontitis include non-surgical treatment and surgical treatment followed by regenerative therapy. Guided tissue regeneration/guided bone regeneration (GTR/GBR) is a clinical procedure that attempts to regenerate the lost periodontal structures, especially the bone. The procedure uses membranes as barriers to prevent gingival epithelium from invading into the bony defect. The transiently stable barrier maintains the space for bone regeneration and integrates with the bone and gingiva overtime. Different types of barrier membranes for GTR/GBR exist on the market.

Most of the commercially available membranes focus exclusively on keeping the gingiva from invading bone space, and these products have numerous drawbacks. Many current membranes only work as physical barriers, and do not provide any bioactivity. The membranes therefore do not allow cell migration, cell penetration, or cell integration. Many membranes are single layered and cannot provide zone-dependent activity. The few membranes that have multiple layers consist of randomly oriented collagen fibers, which provides only a barrier function without spatial guidance. The majority of membrane products in the current market are collagen based. Collagen membranes are expensive, and involves more regulations if the source is from animals or humans. Collagen based membranes degrade quickly and do not match the rate of regeneration of new tissues. Some membrane products include polyglycolic acid (PGA) based scaffolds, but its degradation products cause rapid drops in pH inside the confined space, which can lead to osteolysis. Other membrane products include polylactic acid (PLA), but PLA is a slowly degrading material and usually stays much longer than the regenerated tissue and can almost be considered a non-resorbable material.

Another common oral disease is endodontic disease. The conventional treatment modality is root canal therapy (RCT). Approximately 24 million RCT procedures are performed in the United States each year, with an annual expenditure of 20 to 34 billion dollars. RCT consists of the complete removal of the contaminated pulp tissue from a tooth and filling the root canal space with an inert material.

Conventional RCT has numerous limitations. Current procedures for RCT cannot regenerate new dentin and pulp tissue in teeth, nor can they prevent susceptibility to tooth fracture. Unlike a vital tooth, RCT treated teeth lack the pulp-dentin complex (PDC), a highly organized tissue structure that provides vital neurovascular supply. PDC works as an entity and performs sensation, protection, nutrition, as well as immunologic and neurologic functions. A tooth that has been treated with RCT lacks these important functions. For example, if a secondary carious lesion develops, the tooth will fail to perceive the damage due to the lack of sensory nerves. The lesion may go unnoticed and lead to tooth loss.

Although the reported success rate of RCT on mature teeth is 78-98%, RCT on immature teeth is very challenging. In these cases, the thin dentinal walls of the root and the open apex limit the application of mechanical instrumentation and obturation. Furthermore, conventional RCT cannot promote root development. These cases mostly occur in school-age patients due to traumatic injuries, and most often it involves the anterior teeth, which may significantly affect self-esteem if left untreated.

Regenerative endodontics (RE) is a new modality to treat immature teeth. The primary goal of RE is to promote root development in necrotic immature teeth, and the ultimate goal is to regenerate the healthy vital pulp tissue inside the tooth. In 2014, the American Dental Association made RE mandatory training for endodontic residents in the United States. The current clinical RE protocol includes the disinfection of the root canal followed by inducing bleeding into the canal system through over instrumentation beyond the root apex. The blood delivers stem cells into the root canal, and the blood clot formed inside the root canal provides a scaffold for pulp tissue regeneration. The availability of a stem cell niche that can be accessed by the existing clinical procedure is an advantage that not many tissue engineering fields have. It eliminates the need for delivering exogenous stem cells, which is usually met with technical and ethical debates. The ease of translation and the tremendous clinical relevance has been welcomed and can be judged by the existing literature on the topic.

However, the current challenge for RE is that clinicians face unpredictable outcomes. Furthermore, the histological studies suggest that the regenerated tissues inside the root canal are periodontal tissue, not real pulp dentin tissue. One method to improve RE is by using tissue engineering approaches, which is built upon a combination of scaffolds, growth factor delivery, and stem cells. Several biomaterial scaffolds have been introduced for RE, including platelet rich plasma (PRP) and hydrogels (collagen, hyaluronic acid, polyethylene glycol, and gelatin). However, the major drawback of these scaffolds is that none of them can provide the spatial control that is required for PDC regeneration: angiogenesis and neurogenesis in the center and dentinogenesis in the peripheral area. In addition, scaffolds currently discussed in the literature do not differentially guide cells into specific locations to mimic natural PDC.

There is a need in the art for improved methods of repairing dental tissue damage caused by oral diseases. The present invention meets this need.

### SUMMARY OF THE INVENTION

The present invention relates to biomimetic scaffolds, methods for making the same, and methods for using the same. The scaffolds comprise a plurality of graded or tapered microchannels that provide spatial control for cell penetration. The scaffolds are useful for regenerating missing interface tissue between two adjacent tissues, or regeneration and integration of two adjacent tissues directly.

In one aspect, the invention relates to a biomimetic scaffold material. The biomimetic scaffold material comprises a material having a first surface and a second surface; and a plurality of microchannels extending through the material from the first surface to the second surface. The diameter of the microchannels is tapered, such that the openings to the microchannels from the first surface have a diameter that is greater than the diameter of the openings to the microchannels from the second surface.

In another aspect, the invention relates to a multi-layered biomimetic scaffold material. The multi-layered biomimetic scaffold material comprises at least two layers in contact with each other, each layer comprising a material having a first surface and a second surface; and a plurality of microchannels extending from the first surface to the second surface. The diameter of the microchannels is tapered, such that the openings to the microchannels from the first surface have a diameter that is greater than the diameter of the openings to the microchannels from the second surface.

In one embodiment, the material has a thickness between 10 μm and 10 mm. In one embodiment, the microchannel openings from the first surface have a diameter between 10 and 1000 μm. In one embodiment, the microchannel openings from the second surface have a diameter between 1 and 10 μm. In one embodiment, at least two scaffold materials are combined end-to-end.

In one embodiment, the material is a biodegradable material. In one embodiment, the multi-layered scaffold material has a first layer having a degradation rate that is faster than the at least second layer. In one embodiment, the material comprises poly(lactic-co-glycolic acid) (PLGA). In one embodiment, the scaffold material is cylindrical.

In another aspect, the invention relates to a method of making a biomimetic scaffold. The method comprises the steps of mixing a polymer with a solvent to create a polymer and solvent mixture; casting the polymer and solvent mixture on a glass plate; and submerging the glass plate in an antisolvent bath.

In another aspect, the invention relates to a method of making a multi-layered biomimetic scaffold. The method comprises the steps of making a first layer using the method of making a biomimetic scaffold as described elsewhere herein; making at least one second layer using the method of making a biomimetic scaffold as described elsewhere herein; and securing the at least two layers together.

In one embodiment, the antisolvent bath is changed at set intervals. In one embodiment, the methods further comprise a step of lyophilizing the scaffold to remove excess moisture. In one embodiment, the polymer is PLGA. In one embodiment, the solvent is dimethyl sulfoxide (DMSO). In one embodiment, the antisolvent is water.

In another aspect, the invention relates to method of periodontal ligament regeneration. The method comprises the steps of providing a biomimetic scaffold comprising a first layer and a second layer; and inserting the biomimetic scaffold into the space between a tooth and alveolar bone such that the first layer contacts the cementum of the tooth and the second layer contacts the alveolar bone.

In one embodiment, the first layer of the biomimetic scaffold contacts the cementum with a first surface comprising microchannels having first openings of 50-80 μm and contacts the second layer with a second surface comprising microchannels having second openings of 5-10 μm. In one embodiment, the second layer of the biomimetic scaffold contacts the first layer with a first surface comprising microchannels having first openings of 20-30 μm and contacts the alveolar bone with a second surface comprising microchannels having second openings of less than 5 μm.

In another aspect, the invention relates to a method of endodontic regeneration. The method comprises the steps of providing a biomimetic scaffold having a first layer and a second layer; and inserting the biomimetic scaffold into the root canal space such that the first layer faces the interior of the root canal space and the second layer contacts the dentin.

In one embodiment, the first layer of the biomimetic scaffold faces the interior of the root canal space with a first surface comprising microchannels having first openings of 50-80 μm and contacts the second layer with a second surface comprising microchannels having second openings of 5-10 μm. In one embodiment, the second layer of the biomimetic scaffold contacts the first layer with a first surface comprising microchannels having first openings of 20-30 μm and contacts the dentin with a second surface comprising microchannels having second openings of less than 5 μm.

In another aspect, the invention relates to a method of guided tissue regeneration/guided bone regeneration. The method comprises the steps of providing a biomimetic scaffold having a first surface and a second surface; and inserting the biomimetic scaffold into the alveolar bone defect space such that the first surface faces the bony defect and the second surface contacts the gingiva.

In one embodiment, the first surface of the biomimetic scaffold comprises microchannels having first openings of 20-30 μm. In one embodiment, the second surface of the biomimetic scaffold comprises microchannels having second openings of 5-10 μm.

In another aspect, the invention relates to a kit for repairing tissue. The kit comprises at least one biomimetic scaffold material of the present invention, as described elsewhere herein. In one embodiment, the kit further comprises instructional material for performing the methods of periodontal ligament regeneration, endodontic regeneration, and guided tissue regeneration/guided bone regeneration, as described elsewhere herein. In one embodiment, the at least one biomimetic scaffold material is provided in a preset size.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1 depicts an SEM image of an exemplary scaffold of the present invention.

FIG. 2, comprising FIG. 2A and FIG. 2B, depicts an SEM image and a schematic of an exemplary multilayered scaffold, respectively.

FIG. 3, comprising FIG. 3A and FIG. 3B, depicts a series of schematic representations of differential cell penetration into an exemplary scaffold. FIG. 3A: initial position of cells on the surfaces of a scaffold to be inserted into the interfacial space. FIG. 3B: cell penetration at different stages of culture. Diagrams are based on the measurement by confocal image using MATLAB and OTSU threshold

FIG. 4 depicts a series of schematic presentations of cell penetration at different stages of culturing.

FIG. 5, comprising FIG. 5A through FIG. 5D, depicts a series of SEM images of an exemplary laminated scaffold. FIG. 5A: DPSCs on the surface of the large pores of the permissive side. FIG. 5B: DPSC on the surface of the small pores of the semi-permissive side. FIG. 5C: High magnification of the large pores of the permissive side, with penetration of cells into the pores (arrow). FIG. 5D: Cross section showing DPSC penetration into the pores.

FIG. 6, comprising FIG. 6A through 6C, depicts a series of images illustrating periodontal/alveolar composition. FIG. 6A: anatomical sketch of the periodontal ligament (PDL). FIG. 6B and FIG. 6C: histological cross sections of PDL showing orientation of Sharpeys fibers. ABP: alveolar bone proper; AB: alveolar bone; AEFC: acellular extrinsic fiber cementum. Photo courtesy of: Clinical periodontology and implant dentistry, 5^{th} edition.

FIG. 7, comprising FIG. 7A through 7C, depicts a series of images illustrating periodontal disease and treatment. FIG. 7A: clinical representation of periodontal disease with loss of gingival and bone tissue. FIG. 7B: placement of membranes for guided tissue regeneration/guided bone regeneration (GTR/GBR). FIG. 7C: schematic illustration of GTR. Photos courtesy of: Gore Medical and Atlas of Cosmetic and Reconstructive Periodontal Surgery, 3^{rd} edition.

FIG. 8, comprising FIG. 8A through FIG. 8C, depicts a series of images illustrating cell activity on an exemplary scaffold in (FIG. 8A) the initial stage, (FIG. 8B) the mid stage, and (FIG. 8C) the late stage when used for the regeneration of the PDL.

FIG. 9, comprising FIG. 9A and FIG. 9B, depicts a series of images illustrating an exemplary single layered scaffold used for GTR/GBR. FIG. 9A: scaffold placed between the gingiva and bony defect. FIG. 9B: confocal image of a single layered scaffold for GTR (confocal imaging, stacked cross sections).

FIG. 10, comprising FIG. 10A through FIG. 10C, depicts a series of images illustrating cell activity on an exemplary scaffold in (FIG. 10A) the initial stage, (FIG. 10B) the mid stage, and (FIG. 10C) the late stage when used for the regeneration of the PDC in regenerative endodontics (RE).

FIG. 11, comprising FIG. 11A through FIG. 11D, depicts a series of fluorescence microscopy images demonstrating cell penetration into exemplary scaffolds. FIG. 11A and FIG. 11B: 12% PLGA scaffold. FIG. 11C and FIG. 11D: 20% PLGA scaffold. (Confocal imaging, cross section)

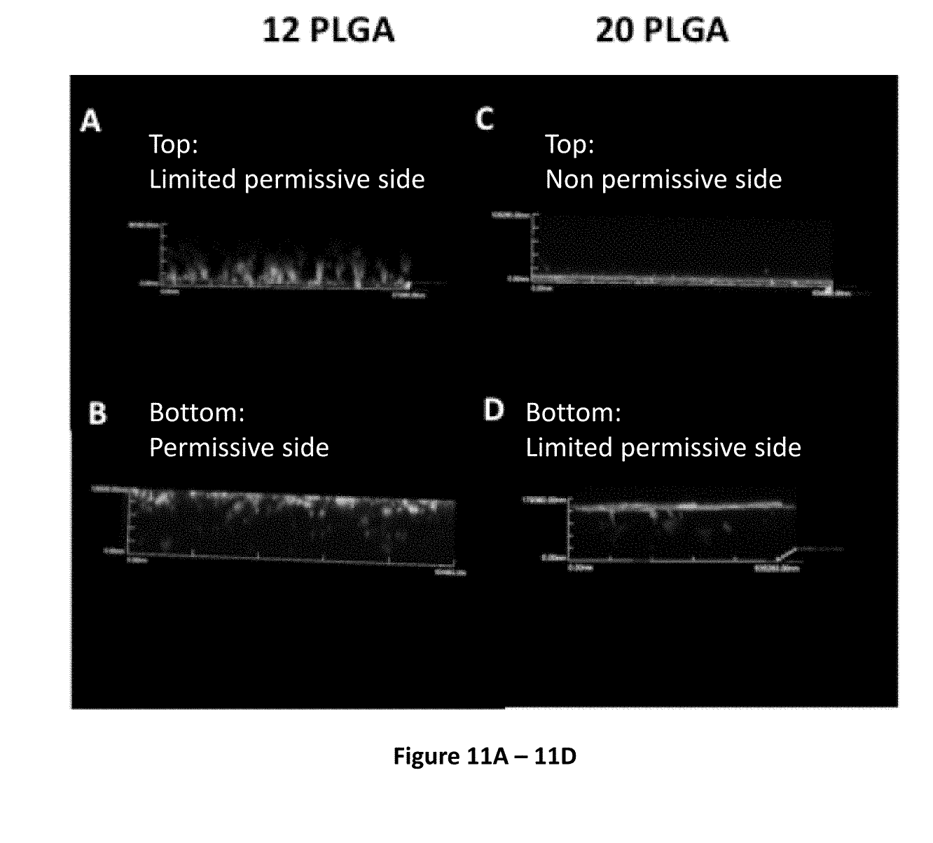

FIG. 12 depicts the results of experiments demonstrating the printing of Vitamin B2 on an exemplary PLGA scaffold using a modified 3D printer.

FIG. 13 illustrates the pulp-dentin complex.

FIG. 14 depicts the results of laminating a 12% w/v PLGA scaffold layer with a 20% w/v PLGA scaffold layer.

FIG. 15 depicts the results of experiments investigating the proliferation and penetration of dental pulp stem cells (DPSCs) cultured on the 12% w/v PLGA scaffold layer at 7 days and 14 days.

FIG. 16 depicts the results of experiments investigating the proliferation and penetration of DPSCs cultured on the 20% w/v PLGA scaffold layer at 7 days and 14 days.

FIG. 17 depicts the results of experiments investigating the pore morphology of various scaffolds after being allowed to degrade in 37° C. PBS at 4 weeks and 8 weeks.

FIG. 18 depicts the results of experiments investigating the penetration of cells into a 12% w/v PLGA scaffold layer and a 20% w/v PLGA scaffold layer over time.

FIG. 19 depicts the results of experiments investigating relationship between cell penetration and scaffold pore diameters.

### DETAILED DESCRIPTION

The present invention provides biomimetic scaffolds, methods for making the same, and methods for using the same. The scaffolds are substantially planar and comprise a plurality of microchannels extending from one surface of a scaffold to the opposite surface of the scaffold. The microchannels have a gradation or a taper, such that the microchannels have a wide diameter at one end and a narrow diameter at the opposite end. The scaffolds can support cell proliferation on all surfaces as well as throughout its interior.

The scaffolds described herein include single layer and multi-layer scaffolds. Multi-layered scaffolds comprise two or more fused scaffolds in a back-to-back or end-to-end arrangement. Multi-layered scaffolds provide spatial control for cells by allowing differential cell penetration into different layers, as well as different scaffold degradation rates for integration with surrounding tissue.

In certain embodiments, the scaffolds described herein are manufactured using diffusion induced phase separation. The methods use a mixture of polymers and solvents with antisolvents to generate the scaffolds of the present invention. The methods are amenable to modification in order to optimize scaffold parameters such as degradation, microchannel diameter; and to incorporate additional components such as growth factors, proteins, and the like.

### Definitions

It is to be understood that the figures and descriptions of the present invention have been simplified to illustrate elements that are relevant for a clear understanding of the present invention, while eliminating, for the purpose of clarity, many other elements found in typical scaffolds. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

Unless defined elsewhere, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, and ±0.1% from the specified value, as such variations are appropriate.

As used herein, an “antisolvent” is a substance in which a solute is substantially not soluble. It should be understood that it is possible that the antisolvent may be capable of dissolving some amount of the solute without departing from the scope of the present invention. The antisolvent is, however, preferably incapable of dissolving a significant portion of the solute such that at least a significant portion of solute is, in effect, not soluble in the antisolvent.

As used here, “biocompatible” refers to any material, which, when implanted in a mammal, does not provoke an adverse response in the mammal. A biocompatible material, when introduced into an individual, is not toxic or injurious to that individual, nor does it induce immunological rejection of the material in the mammal.

As used herein, a “culture,” refers to the cultivation or growth of cells, for example, tissue cells, in or on a nutrient medium. As is well known to those of skill in the art of cell or tissue culture, a cell culture is generally begun by removing cells or tissue from a human or other animal, dissociating the cells by treating them with an enzyme, and spreading a suspension of the resulting cells out on a flat surface, such as the bottom of a Petri dish. There the cells generally form a thin layer of cells called a “monolayer” by producing glycoprotein-like material that causes the cells to adhere to the plastic or glass of the Petri dish. A layer of culture medium, containing nutrients suitable for cell growth, is then placed on top of the monolayer, and the culture is incubated to promote the growth of the cells.

As used herein, “extracellular matrix composition” includes both soluble and non-soluble fractions or any portion thereof. The non-soluble fraction includes those secreted ECM proteins and biological components that are deposited on the support or scaffold. The soluble fraction includes refers to culture media in which cells have been cultured and into which the cells have secreted active agent(s) and includes those proteins and biological components not deposited on the scaffold. Both fractions may be collected, and optionally further processed, and used individually or in combination in a variety of applications as described herein.

As used herein, a “graft” refers to a cell, tissue, organ, or biomaterial that is implanted into an individual, typically to replace, correct or otherwise overcome a defect. A graft may further comprise a scaffold. The tissue or organ may consist of cells that originate from the same individual; this graft is referred to herein by the following interchangeable terms: “autograft”, “autologous transplant”, “autologous implant” and “autologous graft”. A graft comprising cells from a genetically different individual of the same species is referred to herein by the following interchangeable terms: “allograft,” “allogeneic transplant,” “allogeneic implant,” and “allogeneic graft.” A graft from an individual to his identical twin is referred to herein as an “isograft,” a “syngeneic transplant,” a “syngeneic implant” or a “syngeneic graft.” A “xenograft,” “xenogeneic transplant,” or “xenogeneic implant” refers to a graft from one individual to another of a different species. The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

As used herein “growth factors” is intended the following non-limiting factors including, but not limited to, growth hormone, erythropoietin, thrombopoietin, interleukin 3, interleukin 6, interleukin 7, macrophage colony stimulating factor, c-kit ligand/stem cell factor, osteoprotegerin ligand, insulin, insulin like growth factors, epidermal growth factor (EGF), fibroblast growth factor (FGF), nerve growth factor, ciliary neurotrophic factor, platelet derived growth factor (PDGF), transforming growth factor (TGF-beta), hepatocyte growth factor (HGF), and bone morphogenetic protein at concentrations of between picogram/ml to milligram/ml levels.

As used herein, “polymer” includes copolymers. “Copolymers” are polymers formed of more than one polymer precursor. Polymers as used herein include those that are soluble in a solvent that are insoluble in an antisolvent.

As used herein, “scaffold” refers to a structure, comprising a biocompatible material that provides a surface suitable for adherence and proliferation of cells. A scaffold may further provide mechanical stability and support. A scaffold may be in a particular shape or form so as to influence or delimit a three-dimensional shape or form assumed by a population of proliferating cells. Such shapes or forms include, but are not limited to, films (e.g. a form with two-dimensions substantially greater than the third dimension), ribbons, cords, sheets, flat discs, cylinders, spheres, 3-dimensional amorphous shapes, etc.

As used herein, “tissue engineering” refers to the process of generating a tissue ex vivo for use in tissue replacement or reconstruction. Tissue engineering is an example of “regenerative medicine,” which encompasses approaches to the repair or replacement of tissues and organs by incorporation of cells, gene or other biological building blocks, along with bioengineered materials and technologies.

As used herein, the terms “tissue grafting” and “tissue reconstructing” both refer to implanting a graft into an individual to treat or alleviate a tissue defect, such as a lung defect or a soft tissue defect.

“Transplant” refers to a biocompatible lattice or a donor tissue, organ or cell, to be transplanted. An example of a transplant may include but is not limited to skin cells or tissue, bone marrow, and solid organs such as heart, pancreas, kidney, lung and liver.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6, etc., as well as individual numbers within that range, for example, 1, 2, 2.7, 3, 4, 5, 5.3, 6, and any whole and partial increments there between. This applies regardless of the breadth of the range.

### Biomimetic Scaffolds

The present invention provides devices for regenerative tissue therapy. In various embodiments, the devices are scaffolds, including biomimetic scaffolds. The biomimetic scaffolds are porous with microchannels having varying diameters. The biomimetic scaffolds are tunable to control cell infiltration and scaffold degradation. In various embodiments, the biomimetic scaffolds of the present invention may be combined to form a multi-layer scaffold.

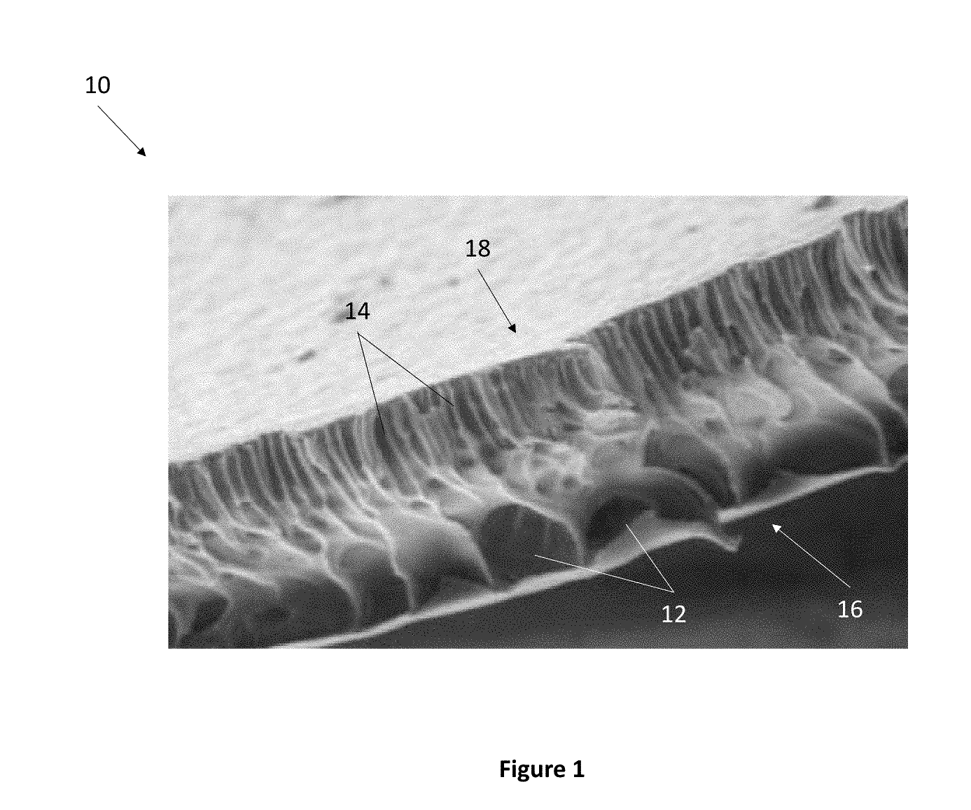

In one embodiment, the present invention relates to a single layer biomimetic scaffold. Referring now to FIG. 1, an exemplary single layer biomimetic scaffold 10 is depicted. A single layer biomimetic scaffold comprises a first surface 16, a second surface 18, and a plurality of microchannels. In certain embodiments, the plurality of microchannels comprise a first opening 12 on the first surface 16 and a second opening 14 on the second surface 18.

The microchannels can have any suitable diameter. In one embodiment, first opening 12 comprises a wide diameter. For example, first opening 12 can have a diameter between 10-1000 μm. In one embodiment, first opening 12 can have a diameter between 50-80 μm. In another embodiment, first opening 12 can have a diameter between 20-30 μm. In one embodiment, second opening 14 comprises a narrow diameter. For example, second opening 14 can have a diameter between 1-100 μm. In one embodiment, second opening 14 can have a diameter between 5-10 μm. In another embodiment, second opening 14 can have a diameter less than 5 μm. The plurality of microchannels are tapered or graded, such that the diameter of the microchannels steadily decreases along the length of the microchannels, between first opening 12 and second opening 14. In one embodiment, the microchannel gradation can be described as a percent reduction in diameter from first opening 12 to second opening 14. For example, the percent reduction in diameter from first opening 12 to second opening 14 can be between 50% and 99%. In various embodiments, the scaffold has a porosity between 50% and 99%. In one embodiment, the scaffold has a porosity between 70% and 90%.

In various embodiments, biomimetic scaffold 10 is substantially planar, such as in the form of a sheet. Biomimetic scaffold 10 can have any suitable thickness. For example, the thickness of biomimetic scaffold 10 can be less than 100 μm to several millimeters. In one embodiment, the thickness of biomimetic scaffold is between 100-300 μm. Biomimetic scaffold 10 can have any geometric shape. In various embodiments, biomimetic scaffold 10 can be trimmed to accommodate any suitable shape. In one embodiment, biomimetic scaffold 10 is rolled into a tube, such that one surface faces the inside of the tube and the opposite surface faces the outside of the tube.

In various embodiments, a plurality of biomimetic scaffolds 10 may be combined to form a multi-component scaffold. For example, a plurality of biomimetic scaffolds 10 may be fused end-to-end to form a larger scaffold comprising a plurality of regions having different microchannel dimensions.

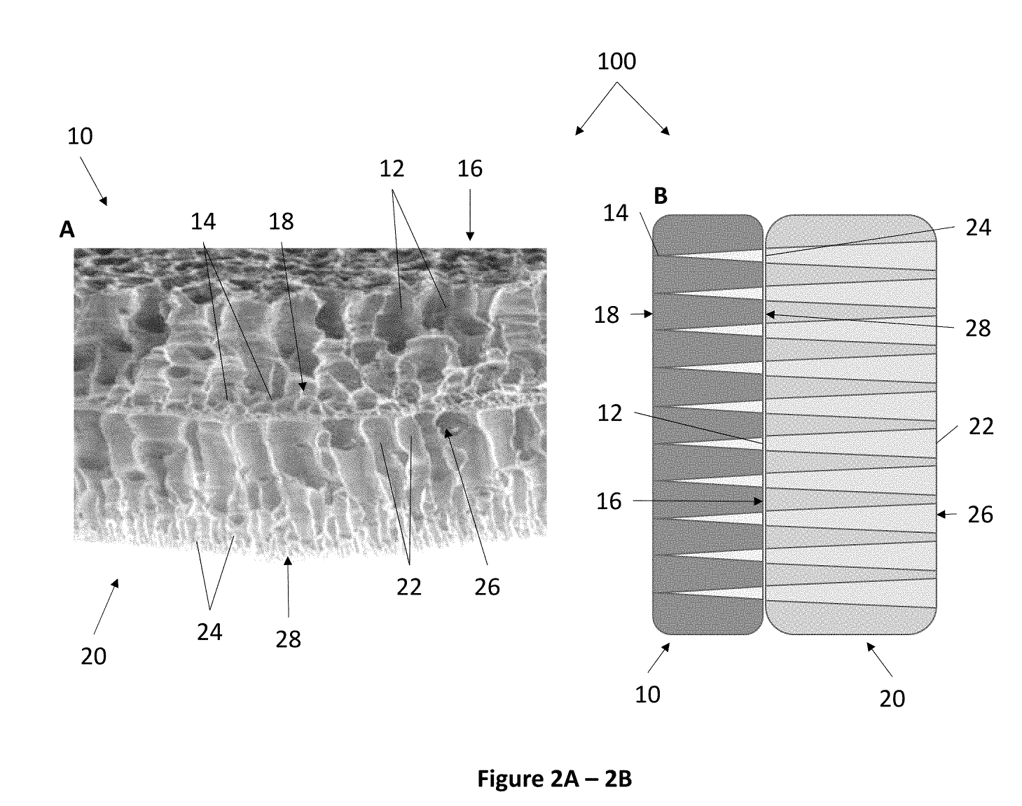

In another embodiment, a plurality of biomimetic scaffolds 10 may be stacked to form a multi-layered scaffold 100, such as in FIG. 2. Multi-layered scaffold 100 provides a plurality of layers, where each layer may have different microchannel dimensions, scaffold composition, degradation rates, and the like. For example, an exemplary multi-layered scaffold may comprise at least a biomimetic scaffold 10 having first openings 12, first surface 16, second openings 14, and second surface 18; and at least a biomimetic scaffold 20 having first openings 22, first surface 26, second openings 24, and second surface 28.

Biomimetic scaffold 10 can be made from any suitable material. For example, biomimetic scaffold 10 can comprise ceramics, polymers, metals, or any combination thereof. In one embodiment, biomimetic scaffold 10 comprises poly(lactic-co-glycolic acid) (PLGA). The material can be biodegradable and non-biodegradable. In various embodiments, the scaffold can comprise materials having different rates of degradation. For example, a rapidly degrading material can support regeneration and integration of a quickly proliferating tissue type, and a slowly degrading material can support regeneration and integration of a slower proliferating tissue type.

In one embodiment, the scaffold may comprise any polysaccharide, including glycosaminoglycans (GAGs) or glucosaminoglycans, with suitable viscosity, molecular mass and other desirable properties. The term “glycosaminoglycan” is intended to encompass any glycan (i.e., polysaccharide) comprising an unbranched polysaccharide chain with a repeating disaccharide unit, one of which is always an amino sugar. These compounds as a class carry a high negative charge, are strongly hydrophilic, and are commonly called mucopolysaccharides. This group of polysaccharides includes heparin, heparan sulfate, chondroitin sulfate, dermatan sulfate, keratan sulfate, and hyaluronic acid. These GAGs are predominantly found on cell surfaces and in the extracellular matrix. The term “glucosaminoglycan” is also intended to encompass any glycan (i.e. polysaccharide) containing predominantly monosaccharide derivatives in which an alcoholic hydroxyl group has been replaced by an amino group or other functional group such as sulfate or phosphate. An example of a glucosaminoglycan is poly-N-acetyl glucosaminoglycan, commonly referred to as chitosan. Exemplary polysaccharides that may be useful in the present invention include dextran, heparan, heparin, hyaluronic acid, alginate, agarose, carageenan, amylopectin, amylose, glycogen, starch, cellulose, chitin, chitosan and various sulfated polysaccharides such as heparan sulfate, chondroitin sulfate, dextran sulfate, dermatan sulfate, or keratan sulfate.

The scaffold may comprise natural materials such as proteins derived from corn, wheat, potato, sorghums, tapioca, rice, arrow root, sago, soybean, pea, sunflower, peanut, gelatin, and the like. Using natural materials also minimizes rejection or immunological responses to an implanted scaffold.

Synthetic materials for use in the scaffold include any materials prepared through any method of artificial synthesis, processing, isolation, or manufacture. The synthetic materials are preferably biologically compatible for administration in vivo or in vitro. Such polymers include but are not limited to the following: poly(urethanes), poly(siloxanes) or silicones, poly(ethylene), poly(vinyl pyrrolidone), poly(2-hydroxy ethyl methacrylate), poly(N-vinyl pyrrolidone), poly(methyl methacrylate), poly(vinyl alcohol), poly(acrylic acid), polyacrylamide, poly(ethylene-co-vinyl acetate), poly(ethylene glycol), poly(methacrylic acid), polylactic acid (PLA), polyglycolic acids (PGA), poly(lactide-co-glycolides) (PLGA), nylons, polyamides, polyanhydrides, poly(ethylene-co-vinyl alcohol) (EVOH), polycaprolactone, poly(vinyl acetate) (PVA), polyvinylhydroxide, poly(ethylene oxide) (PEO) and polyorthoesters or any other similar synthetic polymers that may be developed that are biologically compatible. Polymers with cationic moieties can also be used, such as poly(allyl amine), poly(ethylene imine), poly(lysine), and poly(arginine). The polymers may have any molecular structure including, but not limited to, linear, branched, graft, block, star, comb and dendrimer structures.

In various embodiments, the biomimetic scaffold may also be modified with functional groups for covalently attaching a variety of proteins (e.g., collagen) or compounds such as therapeutic agents. Therapeutic agents which may be linked to the scaffold include, but are not limited to, analgesics, anesthetics, antifungals, antibiotics, anti-inflammatories, anthelmintics, antidotes, antiemetics, antihistamines, antihypertensives, antimalarials, antimicrobials, antipsychotics, antipyretics, antiseptics, antiarthritics, antituberculotics, antitussives, antivirals, cardioactive drugs, cathartics, chemotherapeutic agents, a colored or fluorescent imaging agent, corticoids (such as steroids), antidepressants, depressants, diagnostic aids, diuretics, enzymes, expectorants, hormones, hypnotics, minerals, nutritional supplements, parasympathomimetics, potassium supplements, radiation sensitizers, a radioisotope, sedatives, sulfonamides, stimulants, sympathomimetics, tranquilizers, urinary anti-infectives, vasoconstrictors, vasodilators, vitamins, xanthine derivatives, and the like. The therapeutic agent may also be other small organic molecules, naturally isolated entities or their analogs, organometallic agents, chelated metals or metal salts, peptide-based drugs, or peptidic or non-peptidic receptor targeting or binding agents. It is contemplated that linkage of the therapeutic agent to the scaffold may be via a protease sensitive linker or other biodegradable linkage. Molecules which may be incorporated into the biomimetic scaffold include, but are not limited to, vitamins and other nutritional supplements; glycoproteins (e.g., collagen); fibronectin; peptides and proteins; carbohydrates (both simple and/or complex); proteoglycans; antigens; oligonucleotides (sense and/or antisense DNA and/or RNA); antibodies (for example, to infectious agents, tumors, drugs or hormones); and gene therapy reagents.

In various embodiments, the biomimetic scaffold can further comprise extracellular matrix materials and blends of naturally occurring extracellular matrix materials, including but not limited to collagen, fibrin, fibrinogen, thrombin, elastin, laminin, fibronectin, hyaluronic acid, chondroitin 4-sulfate, chondroitin 6-sulfate, dermatan sulfate, heparin sulfate, heparin, and keratan sulfate, proteoglycans, and combinations thereof. Some collagens that are used include but are not limited to collagen types I, II, III, IV, V, VI, VII, VIII, IX, X, XI, XII, XIII, XIV, XV, XVI, XVII, XVIII, and XIX. These proteins may be in any form, including but not limited to native and denatured forms. The biomimetic scaffold can further comprise carbohydrates such as polysaccharides (e.g. cellulose and its derivatives), chitin, chitosan, alginic acids, and alginates such as calcium alginate and sodium alginate. These materials may be isolated from plant products, humans or other organisms or cells or synthetically manufactured. Also contemplated are crude extracts of tissue, extracellular matrix material, or extracts of non-natural tissue, alone or in combination. Extracts of biological materials, including but are not limited to cells, tissues, organs, and tumors may also be included.

In one embodiment, the scaffold can further comprise natural or synthetic drugs, such as nonsteroidal anti-inflammatory drugs (NSAIDs). In one embodiment, the scaffold can further comprise antibiotics, such as penicillin. In one embodiment, the scaffold can further comprise natural peptides, such as glycyl-arginyl-glycyl-aspartyl-serine (GRGDS), arginylglycylaspartic acid (RGD), and amelogenin. In one embodiment, the scaffold can further comprise proteins, such as soy, chitosan, and silk. In one embodiment, the scaffold can further comprise polysaccharides, such as sucrose, fructose, cellulose, mannitol, and chondroitin sulfate. In one embodiment, the scaffold can further comprise extracellular matrix proteins, such as fibronectin, dentin matrix protein, vitronectin, laminin, collagens, and vixapatin (VP12). In one embodiment, the scaffold can further comprise disintegrins, such as VLO4. In one embodiment, the scaffold can further comprise decellularized or demineralized tissue, such as enamel, dentin, and bone. In one embodiment, the scaffold can further comprise synthetic peptides, such as emdogain. In one embodiment, the scaffold can further comprise polymers, such as polycaprolactone, polyethylene glycols, poly vinyl alcohol, poly lactides, and poly glycolides. In one embodiment, the scaffold can further comprise natural bioceramic, such as natural hydroxyapatite and enamel/bone/dentin fragments. In one embodiment, the scaffold can further comprise synthetic bioceramic, such as synthetic hydroxyapatite, nanodiamonds, b-tricalcium phosphate, and calcium sulfates. In one embodiment, the scaffold can further comprise bioactive glasses, such as bioglass and perioglass. In one embodiment, the scaffold can further comprise nutrients, such as bovine serum albumin. In one embodiment, the scaffold can further comprise vitamins, such as vitamin B2, vitamin Ad, Vitamin D, Vitamin E, and Vitamin K. In one embodiment, the scaffold can further comprise nucleic acids, such as mRNA and DNA. In one embodiment, the scaffold can further comprise natural or synthetic steroids and hormones, such as dexamethasone, hydrocortisone, estrogens, and its derivatives. In one embodiment, the scaffold can further comprise growth factors, such as fibroblast growth factor (FGF), transforming growth factor beta (TGF-β), and epidermal growth factor (EGF). In one embodiment, the scaffold can further comprise a delivery vehicle, such as nanoparticles, microparticles, liposomes, viral and non-viral transfection systems.

In one embodiment, the scaffold is cell-free. In certain embodiments, upon implantation, the scaffold supports cell migration and proliferation from native tissue. In another embodiment, the scaffold is seeded with one or more populations of cells to form an artificial tissue construct. The artificial tissue construct may be autologous, where the cell populations are derived from a patient's own tissue, or allogenic, where the cell populations are derived from another subject within the same species as the patient. The artificial organ construct may also be xenogenic, where the different cell populations are derived form a mammalian species that is different from the subject. For example the cells may be derived from organs of mammals such as humans, monkeys, dogs, cats, mice, rats, cows, horses, pigs, goats and sheep.

Cells may be isolated from a number of sources, including, for example, biopsies from living subjects and whole-organ recover from cadavers. The isolated cells are preferably autologous cells, obtained by biopsy from the subject intended to be the recipient. The biopsy may be obtained using a biopsy needle, a rapid action needle which makes the procedure quick and simple.

Cells may be isolated using techniques known to those skilled in the art. For example, the tissue may be disaggregated mechanically and/or treated with digestive enzymes and/or chelating agents that weaken the connections between neighboring cells making it possible to disperse the tissue into a suspension of individual cells without appreciable cell breakage. Enzymatic dissociation may be accomplished by mincing the tissue and treating the minced tissue with any of a number of digestive enzymes either alone or in combination. These include but are not limited to trypsin, chymotrypsin, collagenase, elastase, and/or hyaluronidase, DNase, pronase and dispase. Mechanical disruption may also be accomplished by a number of methods including, but not limited to, scraping the surface of the tissue, the use of grinders, blenders, sieves, homogenizers, pressure cells, or sonicators.

Once the tissue has been reduced to a suspension of individual cells, the suspension may be fractionated into subpopulations from which the cells elements may be obtained. This also may be accomplished using standard techniques for cell separation including, but not limited to, cloning and selection of specific cell types, selective destruction of unwanted cells (negative selection), separation based upon differential cell agglutinability in the mixed population, freeze-thaw procedures, differential adherence properties of the cells in the mixed population, filtration, conventional and zonal centrifugation, centrifugal elutriation (counterstreaming centrifugation), unit gravity separation, countercurrent distribution, electrophoresis and fluorescence-activated cell sorting.

Cell fractionation may also be desirable, for example, when the donor has diseases such as cancer or metastasis of other tumors to the desired tissue. A cell population may be sorted to separate malignant cells or other tumor cells from normal noncancerous cells. The normal noncancerous cells, isolated from one or more sorting techniques, may then be used for tissue reconstruction.

Isolated cells may be cultured in vitro to increase the number of cells available for seeding the biomimetic scaffold. The use of allogenic cells, and more preferably autologous cells, is preferred to prevent tissue rejection. However, if an immunological response does occur in the subject after implantation of the artificial organ, the subject may be treated with immunosuppressive agents such as cyclosporin or FK506 to reduce the likelihood of rejection. In certain embodiments, chimeric cells, or cells from a transgenic animal, may be seeded onto the biocompatible scaffold.

Isolated cells may be transfected prior to coating with genetic material. Useful genetic material may be, for example, genetic sequences which are capable of reducing or eliminating an immune response in the host. For example, the expression of cell surface antigens such as class I and class II histocompatibility antigens may be suppressed. This may allow the transplanted cells to have reduced chances of rejection by the host. In addition, transfection could also be used for gene delivery.

Isolated cells may be normal or genetically engineered to provide additional or normal function. Methods for genetically engineering cells with retroviral vectors, polyethylene glycol, or other methods known to those skilled in the art may be used. These include using expression vectors which transport and express nucleic acid molecules in the cells. (See Goeddel; Gene Expression Technology: Methods in Enzymology 185, Academic Press, San Diego, Calif. (1990). Vector DNA may be introduced into prokaryotic or cells via conventional transformation or transfection techniques. Suitable methods for transforming or transfecting host cells can be found in Sambrook et al. (Molecular Cloning: A Laboratory Manual, 3nd Edition, Cold Spring Harbor Laboratory press (2001)), and other laboratory textbooks.

Seeding of cells onto the scaffold may be performed according to standard methods. For example, the seeding of cells onto polymeric substrates for use in tissue repair has been reported (see, e.g., Atala, A. et al., J. Urol. 148(2 Pt 2): 658-62 (1992); Atala, A., et al. J. Urol. 150 (2 Pt 2): 608-12 (1993)). Cells grown in culture may be trypsinized to separate the cells, and the separated cells may be seeded on the scaffold. Alternatively, cells obtained from cell culture may be lifted from a culture plate as a cell layer, and the cell layer may be directly seeded onto the scaffold without prior separation of the cells.

In one embodiment, a range of 1 million to 50 million cells are suspended in medium and applied to each square centimeter of a surface of a scaffold. The scaffold is incubated under standard culturing conditions, such as, for example, 37° C. 5% CO₂, for a period of time until the cells become attached. However, it will be appreciated that the density of cells seeded onto the scaffold may be varied. For example, greater cell densities promote greater tissue regeneration by the seeded cells, while lesser densities may permit relatively greater regeneration of tissue by cells infiltrating the graft from the host. Other seeding techniques may also be used depending on the matrix or scaffold and the cells. For example, the cells may be applied to the matrix or scaffold by vacuum filtration. Selection of cell types, and seeding of cells onto a scaffold, will be routine to one of ordinary skill in the art in light of the teachings herein.

In one embodiment, the scaffold is seeded with one population of cells to form an artificial tissue construct. In another embodiment, the scaffold is seeded on two sides with two different populations of cells. This may be performed by first seeding one side of the scaffold and then seeding the other side. For example, the scaffold may be placed with one side on top and seeded. The scaffold may then be repositioned so that a second side is on top. The second side may then be seeded with a second population of cells. Alternatively, both sides of the scaffold may be seeded at the same time. For example, two cell chambers may be positioned on both sides (i.e., a sandwich) of the scaffold. The two chambers may be filled with different cell populations to seed both sides of the scaffold simultaneously. The sandwiched scaffold may be rotated, or flipped frequently to allow equal attachment opportunity for both cell populations.

In another embodiment, two separate scaffolds may be seeded with different cell populations. After seeding, the two scaffolds may be attached together to form a single scaffold with two different cell populations on the two sides. Attachment of the scaffolds to each other may be performed using standard procedures such as fibrin glue, liquid co-polymers, sutures, and the like.

In order to facilitate cell growth on the scaffold of the present invention, the scaffold may be coated with one or more cell adhesion-enhancing agents. These agents include but are not limited to collagen, laminin, and fibronectin. The scaffold may also contain cells cultured on the scaffold to form a target tissue substitute. In the alternative, other cells may be cultured on the scaffold of the present invention. These cells include, but are not limited to, cells cultured on the scaffold to form alveolar bone, dental pulp cells cultured on the scaffold to form dental pulp tissue, gum tissue cells cultured on the scaffold to form gum tissue, and mixtures thereof.

### Methods of Manufacture

The invention relates to methods of making the biomimetic scaffolds of the present invention. In one embodiment, the methods are useful for making a single layer biomimetic scaffold. In another embodiment, the methods are useful for making a multi-layer biomimetic scaffold. In various embodiments, the methods are useful for making biomimetic scaffolds augmented with any suitable component.

In one embodiment, the methods of making the biomimetic scaffold uses diffusion induced phase separation (DIPS). The methods provide a polymer mixed with a solvent. The concentration of polymer in the mixture can be any suitable concentration. For example, the polymer concentration can be between 1% w/v to 99% w/v. In one embodiment, the polymer concentration is about 5% w/v to about 50% w/v. In one embodiment, the polymer concentration is about 10% w/v to about 30% w/v. In one embodiment, the polymer concentration is about 12% w/v. In another embodiment, the polymer concentration is about 20% w/v.

The polymer and solvent mixture is casted onto a substrate, and the substrate is submerged in an antisolvent where the polymer and solvent mixture is allowed to invert by way of diffusion. In some embodiments, the antisolvent is exchanged at set periods of time. During the inversion process, graded microchannels are formed within the mixture, wherein the wide diameter opening of the microchannels form on the surface contacting the substrate and wherein the narrow diameter opening of the microchannels form on the surface contacting the antisolvent. When inversion is complete, the scaffold is removed from the substrate and lyophilized to remove excess moisture.

Suitable polymers include, but are not limited to: poly(lactic-co-glycolic acid) (PLGA), polycaprolactone, cellulose, polystyrene, poly ethylene glycols, poly lactides, and natural molecules such as soy, chitosan, collagen, and silk. Suitable solvents include, but are not limited to: dimethyl sulfoxide (DMSO), acetic acid, tetrahydrofuran, deimethyl formamide, and the like. Suitable antisolvents include, but are not limited to: water, ammonia, ethylene, benzene, methanol, ethanol, isopropanol, isobutanol, fluorocarbons (including chlorotrifluoromethane, monofluoromethane, hexafluoraethane and 1,1-difluoroethylene), toluene, pyridine, cyclohexane, m-cresol, decalin, cyclohexanol, o-xylene, tetralin, aniline, acetylene, chlorotrifluorosilane, sulfur hexafluoride, and the like. Preferably, the polymer is soluble in the solvent but not soluble in the antisolvent, and preferably, the solvent is miscible with the antisolvent.

In one embodiment, the polymer is PLGA and the solvent is DMSO. In one embodiment, PLGA is solvated in DMSO at 12% w/v. In another embodiment, PLGA is solvated in DMSO at 20% w/v. The PLGA and DMSO mixture is cast on a glass plate substrate, and the glass plate is submerged in a bath comprising water as the antisolvent. In one embodiment, the water is deionized water. In another embodiment, the water is distilled water. The water bath is changed every 6 to 10 hours. In various embodiments, the water bath is changed at least four times. Longer intervals between water bath changes increases the total time of fabrication. In some embodiments, the total time required for the water bath immersion step is between 48 and 72 hours. The scaffold is then removed from the water bath and separated from the glass plate

In one embodiment, the method comprises lyophilizing the scaffold to remove excess moisture. Lyophilization, or freeze-dying, of the scaffold may be carried out by any method known in the art; see, e.g., U.S. Pat. No. 4,001,944. For example, the scaffold may be quickly frozen in 100% ethanol and dry ice, then lyophilized at −20° C. in a sterile lyophilizer until dry.

In one embodiment, the method is amenable to making multi-layer biomimetic scaffolds. For example, prior to the lyophilization step, two or more scaffolds made by this method are laminated together using residual DMSO to bond the layers. In one embodiment, the two or more scaffolds are oriented such that the microchannel are graded in the same direction. For example, a multi-layer biomimetic scaffold comprises two or more single layer biomimetic scaffolds wherein the surface of one scaffold comprising the wide diameter microchannel openings contacts the surface of a second scaffold comprising the narrow diameter microchannel openings.

The methods of making the biomimetic scaffolds of the present invention are amenable to modification to tune the scaffold properties. In one embodiment, the polymer to solvent ratio of the polymer and solvent mixture may be adjusted to tune microchannel dimensions. For example, increasing the ratio of polymer in the polymer and solvent mixture decreases the dimensions of the microchannels. In one embodiment, the solvent used in the polymer and solvent mixture may be changed to tune the microchannel dimensions. For example, a solvent that is less miscible with water will increase the time needed to complete the DIPS procedure, but will produce a scaffold having microchannels with a more gradual taper.

In one embodiment, additives may be added to the antisolvent, which changes diffusion coefficients and thereby modifies the biomimetic scaffold. For example, additional solvent may be added to the antisolvent to reduce the diffusion rate of solvent from the polymer and solvent mixture, and produces a scaffold having microchannels with a more gradual taper.

In another embodiment, porogens may be added to the polymer and solvent mixture to introduce tertiary porosity to the scaffolds. The concentration of porogens may be tuned to create pores that may or may not be interconnected. In one embodiment, the porogens are soluble in water. In various embodiments, the porogens include, but are not limited to: polymers, such as polyvinyl alcohol and polyethylene glycol; sugars, such as dextrose and mannose; and salts, such as sodium chloride.

The methods of making the biomimetic scaffolds of the present invention are amenable to modification to augment the scaffold with additional components. In one embodiment, the scaffold can be augmented with natural or synthetic drugs, such as nonsteroidal anti-inflammatory drugs (NSAIDs). In one embodiment, the scaffold can be augmented with antibiotics, such as penicillin. In one embodiment, the scaffold can be augmented with natural peptides, such as glycyl-arginyl-glycyl-aspartyl-serine (GRGDS), arginylglycylaspartic acid (RGD), and amelogenin. In one embodiment, the scaffold can be augmented with proteins, such as soy, chitosan, and silk. In one embodiment, the scaffold can be augmented with polysaccharides, such as sucrose, fructose, cellulose, mannitol, and chondroitin sulfate. In one embodiment, the scaffold can be augmented with extracellular matrix proteins, such as fibronectin, dentin matrix protein, vitronectin, laminin, collagens, and vixapatin (VP12). In one embodiment, the scaffold can be augmented with disintegrins, such as VLO4. In one embodiment, the scaffold can be augmented with decellularized or demineralized tissue, such as enamel, dentin, and bone. In one embodiment, the scaffold can be augmented with synthetic peptides, such as emdogain. In one embodiment, the scaffold can be augmented with polymers, such as polycaprolactone, polyethylene glycols, poly vinyl alcohol, poly lactides, and poly glycolides. In one embodiment, the scaffold can be augmented with natural bioceramic, such as natural hydroxyapatite and enamel/bone/dentin fragments. In one embodiment, the scaffold can be augmented with synthetic bioceramic, such as synthetic hydroxyapatite, nanodiamonds, b-tricalcium phosphate, and calcium sulfates. In one embodiment, the scaffold can be augmented with bioactive glasses, such as bioglass and perioglass. In one embodiment, the scaffold can be augmented with nutrients, such as bovine serum albumin. In one embodiment, the scaffold can be augmented with vitamins, such as vitamin B2, vitamin Ad, Vitamin D, Vitamin E, and Vitamin K. In one embodiment, the scaffold can be augmented with nuclear acids, such as mRNA and DNA. In one embodiment, the scaffold can be augmented with natural or synthetic steroids and hormones, such as dexamethasone, hydrocortisone, estrogens, and its derivatives. In one embodiment, the scaffold can be augmented with growth factors, such as fibroblast growth factor (FGF), transforming growth factor beta (TGF-β), and epidermal growth factor (EGF). In one embodiment, the scaffold can be augmented with a delivery vehicle, such as nanoparticles, microparticles, liposomes, viral and non-viral transfection systems.

In certain embodiments, the methods of making the biomimetic scaffolds may be augmented with geometric modifications. For example, a graded modification step via controlled sodium hydroxide deposition on the scaffold can expose functional carboxylic acid groups. The graded modification facilitates further covalent bonding of growth factors or growth factory delivery systems.

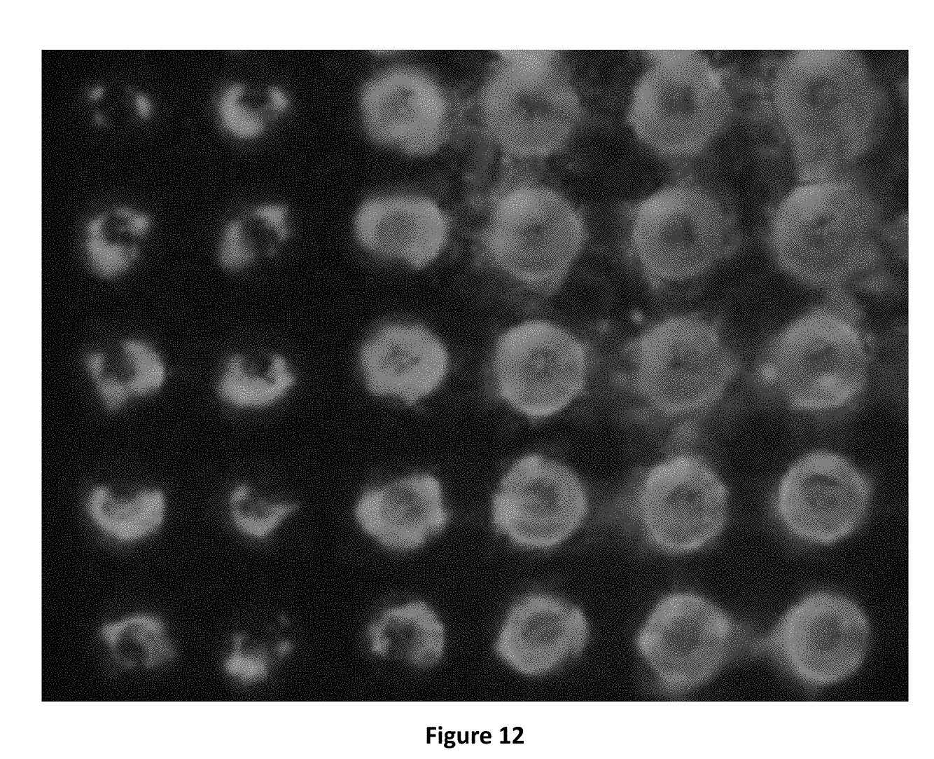

In certain embodiments, the methods of making the biomimetic scaffolds may be augmented with surface modifications. For example, a surface treatment step with cold plasma can increase cell attachment or allow passive adsorption of other molecules. Other surface modifications known in the art may be used to allow passive adsorption of molecules, drugs, growth factors, and the like. In one embodiment, the scaffold surface can be modified to allow additional control of physical dynamics or cellular activities. For example, bioactive molecules may be bioprinted on the scaffold in even or graded fashions, such as in FIG. 12.

### Methods of Use

The invention relates to methods of using the biomimetic scaffolds of the present invention. In another embodiment, the methods are useful for periodontal ligament regeneration. In another embodiment, the methods are useful for regenerative endodontics. In one embodiment, the methods are useful for guided tissue regeneration and guided bone regeneration.

In various embodiments, methods of using the biomimetic scaffolds are amenable to regenerating other tissue. Non-limiting examples of applications of the biomimetic scaffolds include: dental pulp capping for pinpoint pulp exposure in deep carious lesions; guided bone regeneration in craniofacial and other non-stress bearing defects such as calvarial defects; regeneration of any defect located at the interface between at least two different tissue types; and regeneration of any membrane comprising one or more layers.

Periodontal Ligament Regeneration

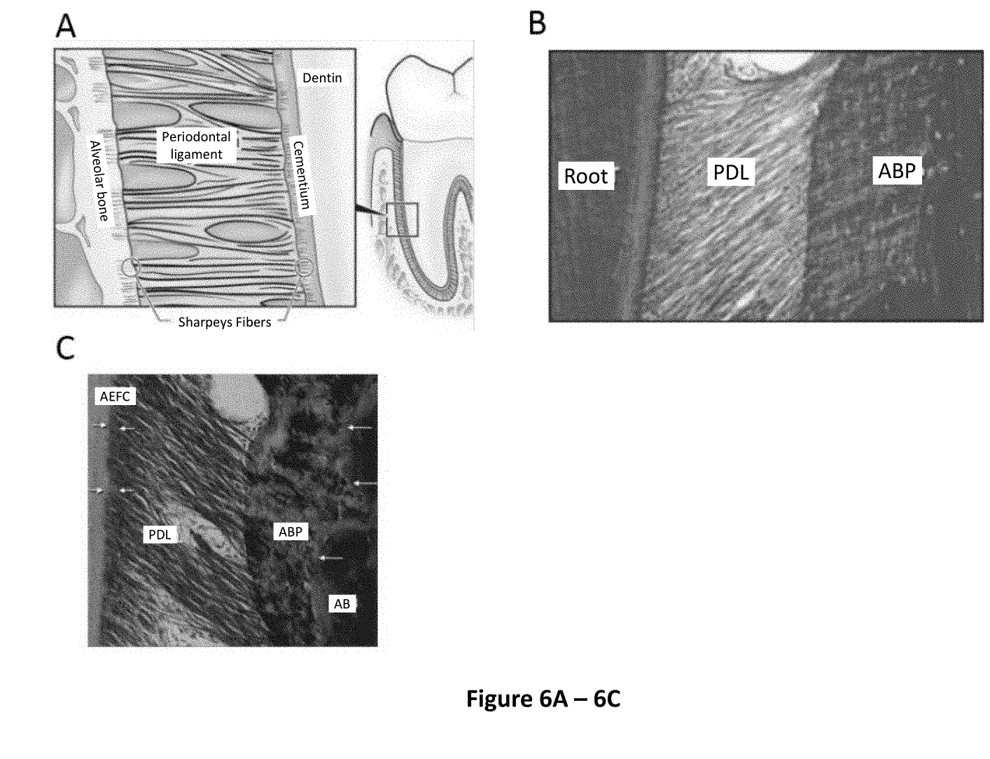

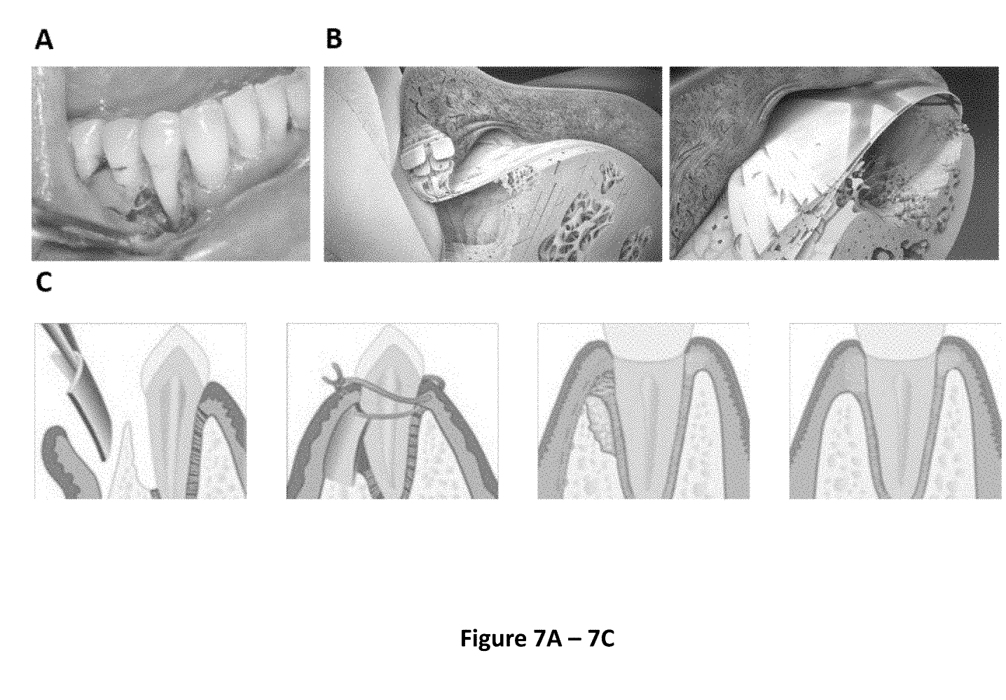

In one embodiment, the invention relates to methods of using biomimetic scaffolds for periodontal ligament (PDL) regeneration (FIG. 6 and FIG. 7). The PDL is a highly organized tissue that connects tooth to the underlying alveolar bone. The method provides spatial guidance for the PDL to regenerate and to allow a matured PDL to integrate with the alveolar bone in periodontal defects. The method prevents the alveolar bone from invading the PDL space, and thereby prevents possible ankyloses to support the regeneration of functional periodontium.

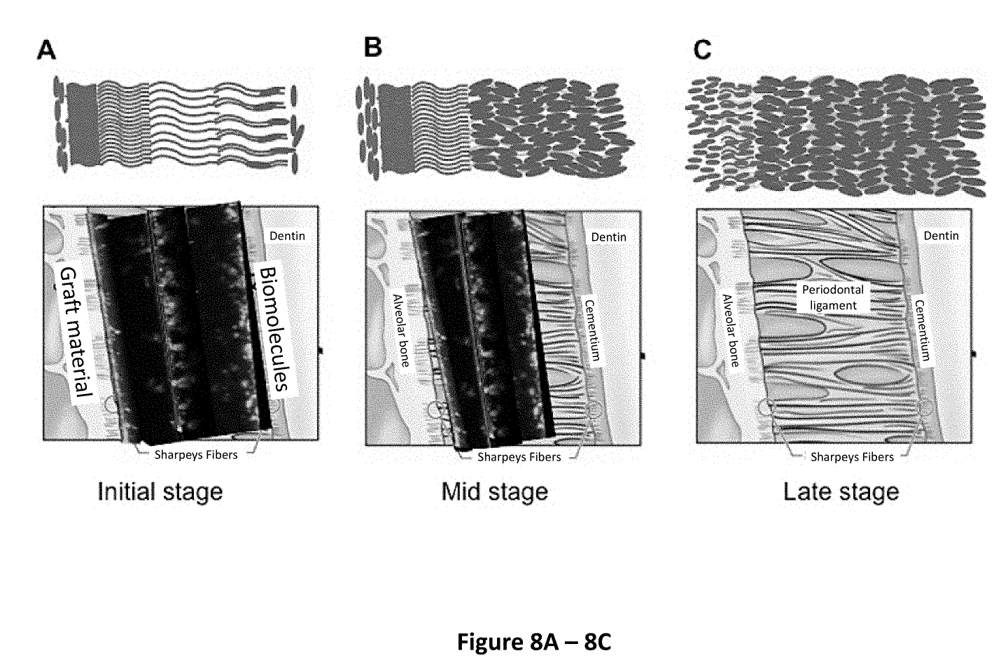

The method provides a multi-layer PDL scaffold having at least two layers that is inserted into the damaged space between a tooth and alveolar bone. A first PDL scaffold layer faces the cementum of a tooth's root surface and supports the regeneration of the PDL (FIG. 8). A second PDL scaffold layer faces the alveolar bone and supports the regeneration of alveolar bone (FIG. 8).

The first PDL scaffold layer comprises microchannels having a first opening of 50-80 μm and a second opening of 5-10 μm. A first surface of the first PDL scaffold layer comprising the microchannel first openings contacts the cementum of the root surface, and a second surface of the first PDL scaffold layer having the second openings contacts the second PDL scaffold layer.

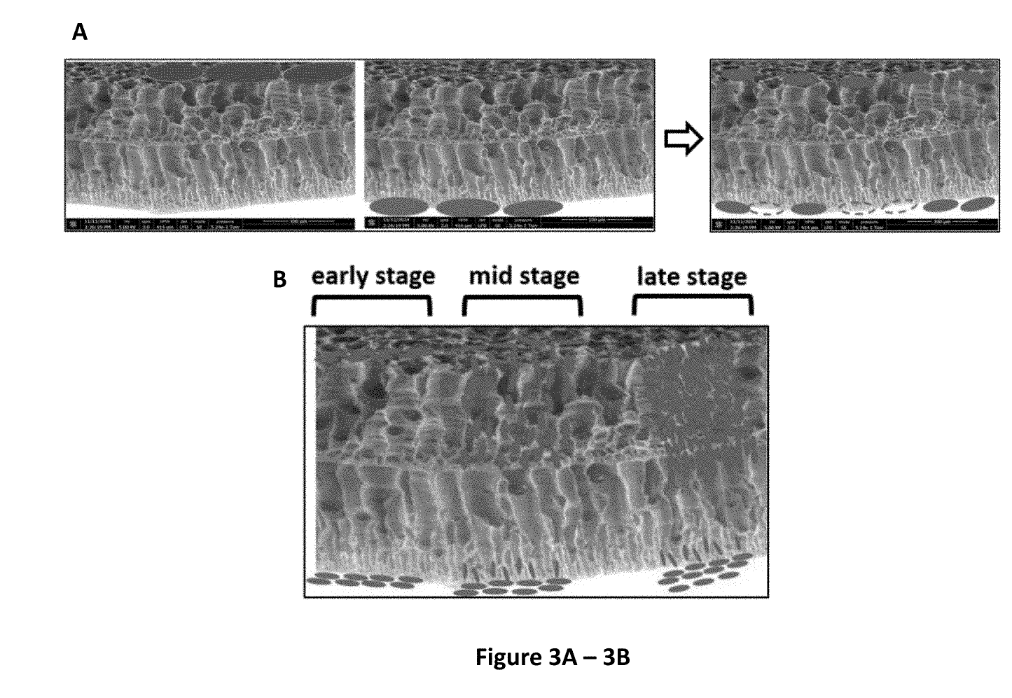

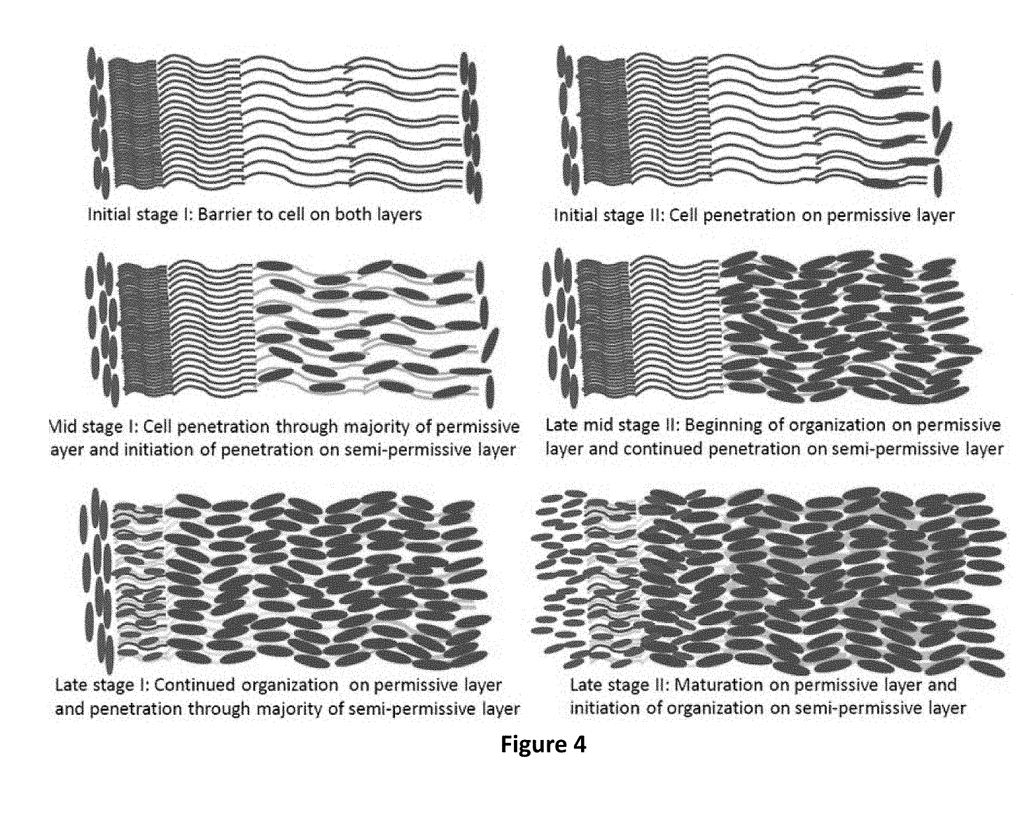

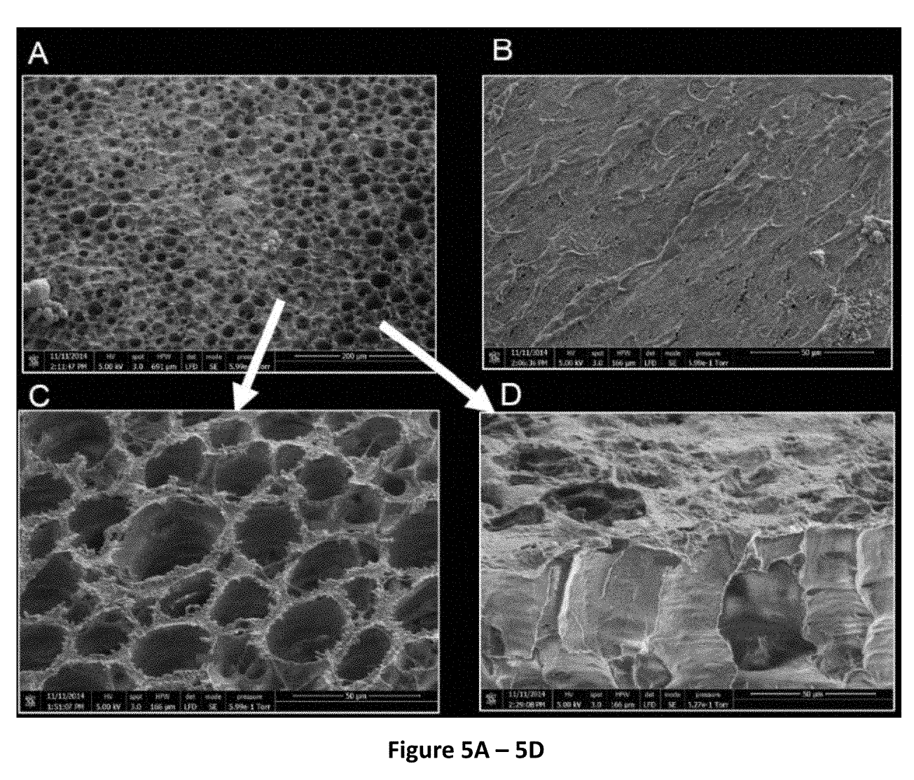

The first surface of the first PDL scaffold layer supports PDL cell attachment, proliferation, and penetration (FIG. 3, FIG. 5, and FIG. 8). The orientation of the microchannels guides cell penetration towards the alveolar bone (FIG. 3 and FIG. 4). The gradation of the microchannels allows the first PDL scaffold layer to support increasing PDL cell attachment, proliferation, and penetration over time (FIG. 3 and FIG. 8). Preferably, the first PDL scaffold layer degrades at a faster rate. A faster rate of degradation permits the PDL forming cells to lay down matrices, organize, and mature prior to integration with alveolar bone (FIG. 3 and FIG. 8).

The second PDL scaffold layer comprises microchannels having a first opening of 20-30 μm and a second opening of less than 5 μm. A first surface of the second PDL scaffold layer comprising the microchannel first openings contacts the first PDL scaffold layer, and a second surface of the second PDL scaffold layer comprising the second openings contacts the alveolar bone (FIG. 8).

The second surface of the second PDL scaffold layer supports bone forming cell attachment and proliferation, but limits penetration. Preferably, the second PDL scaffold layer degrades at a slower rate. A slower rate of degradation prevents early invasion of bone forming cells into the PDL space while still allowing bone regeneration on the scaffold surface (FIG. 3 and FIG. 8).

In certain embodiments, both the first and second PDL scaffold layers support PDL cell attachment, proliferation, and penetration. For example, the PDL biomimetic scaffold is placed in the PDL space between the cementum and alveolar bone, and PDL cells proliferate on the first surface of the first PDL scaffold layer as well as the second surface of the second PDL scaffold layer. In this manner, the developing PDL may form between the scaffold and the bone.

In various embodiments, the PDL biomimetic scaffold may be used in conjunction with osteoinductive or osteoconductive materials. For example, if the alveolar bone defect is large, the second PDL scaffold layer may be augmented with a larger structure comprising materials such as calcium phosphate, calcium sulfate, apatites, bioactive glasses, and the like.

Regenerative Endodontics

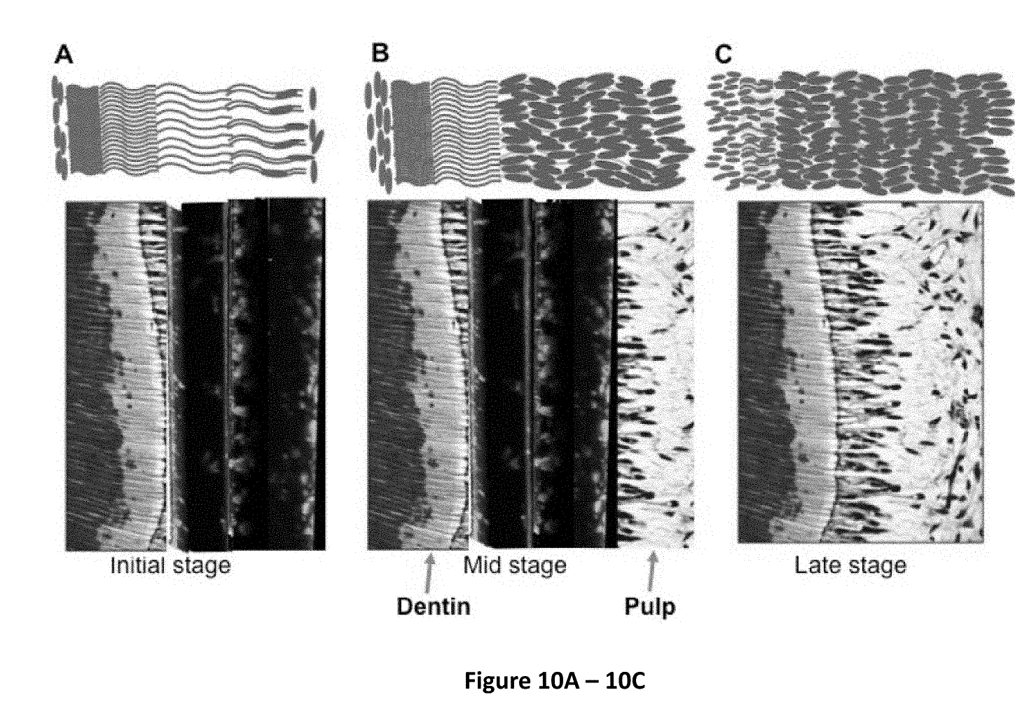

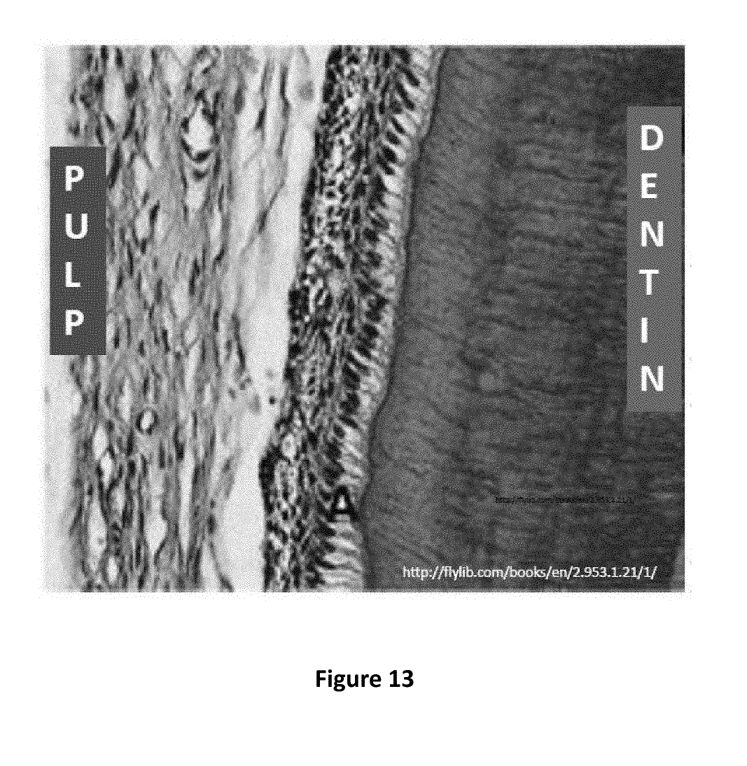

In one embodiment, the invention relates to methods of using biomimetic scaffolds for regenerative endodontics (RE). The method provides guidance for pulp-dentin complex regeneration inside a prepared root canal and provides spatial guidance for cells to form the pulp in the center of the tooth and to integrate with the peripheral regenerating dentin near the walls of the root canal (FIG. 10 and FIG. 13).

The method provides a multi-layer RE scaffold having at least two layers that is inserted into the root canal space. A first RE scaffold layer faces the interior of the root canal space and supports the regeneration of the pulp (FIG. 10). A second RE scaffold layer faces the dentin and supports the regeneration of dentin (FIG. 10).

The first RE scaffold layer comprises microchannels having a first opening of 50-80 μm and a second opening of 5-10 μm. A first surface of the first RE scaffold layer comprising the microchannel first openings faces the interior of the root canal space and contacts remaining pulp tissue or newly introduced tissue (FIG. 10). A second surface of the first RE scaffold layer comprising the second openings contacts the second RE scaffold layer (FIG. 10).

The first surface of the first RE scaffold layer supports dental pulp cell or dental pulp stem cell attachment, proliferation, and penetration (FIG. 3, FIG. 5, and FIG. 10). The orientation of the microchannels guides dental pulp cell penetration towards the adjacent dentin layer (FIG. 3). The gradation of the microchannels allows the first RE scaffold layer to support increasing dental pulp cell attachment, proliferation, and penetration over time. Preferably, the first RE scaffold layer degrades at a faster rate. A faster rate of degradation enhances dental pulp regeneration prior to integration with the dentin layer (FIG. 3 and FIG. 10).

The second RE scaffold layer comprises microchannels having a first opening of 20-30 μm and a second opening of less than 5 μm. A first surface of the second RE scaffold layer comprising microchannel first openings contacts the first RE scaffold layer, and a second surface of the second RE scaffold layer comprising second openings contacts the dentin layer (FIG. 10).

The second surface of the second RE scaffold layer supports dentin forming cell attachment and proliferation, but limits penetration. Preferably, the second RE scaffold layer degrades at a slower rate. A slower rate of degradation prevents early invasion of odontoblasts into the root canal space while still allowing dentin regeneration on the scaffold surface (FIG. 3 and FIG. 10). In conjunction with the orientation of the microchannels, the second RE scaffold layer eventually allows integration of the regenerating pulp tissue with the regenerated dentin layer.

Guided Tissue Regeneration/Guided Bone Regeneration

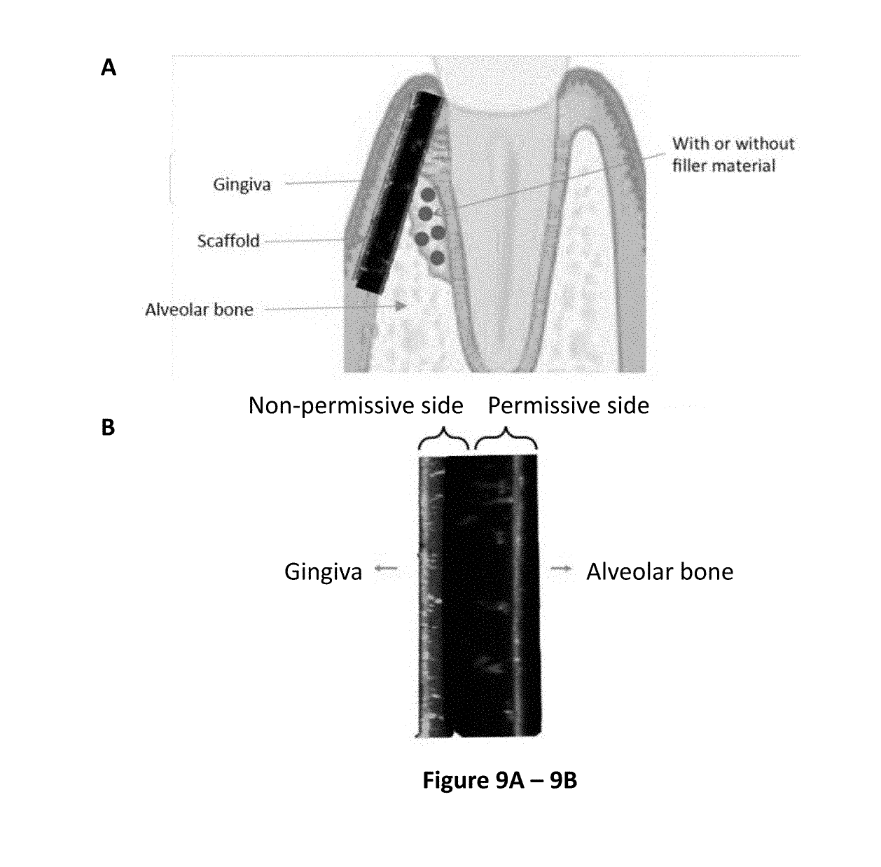

In one embodiment, the invention relates to methods of using biomimetic scaffolds for guided tissue regeneration and guided bone regeneration (GTR/GBR). The method prevents gingival epithelial tissue migration into alveolar bone space and maintains the alveolar bone space for bone regeneration during regenerative periodontal therapy (FIG. 7 and FIG. 9).

The method provides a single layer GTR/GBR scaffold that is inserted into the alveolar bone defect space. A first surface of the GTR/GBR scaffold faces the alveolar bone defect and supports the regeneration of alveolar bone (FIG. 9). A second surface of the GTR/GBR scaffold faces the gums and supports the regeneration of gingival tissue (FIG. 9).

The first surface of the GTR/GBR scaffold comprises microchannels having a first opening of 20-30 μm. The second surface of the GTR/GBR scaffold comprises microchannels having a second diameter of 5-10 μm. The first surface of the GTR/GBR scaffold faces the bony defect (FIG. 9). The second surface of the GTR/GBR scaffold contacts the gingiva (FIG. 9).

The first surface of the GTR/GBR scaffold supports bone forming cell attachment and proliferation (FIG. 3). The orientation of the microchannels guides osteoblast penetration towards the adjacent gingival tissue (FIG. 3 and FIG. 4). The gradation of the microchannels allows the first surface of the GTR/GBR scaffold to support increasing osteoblast cell attachment, proliferation, and penetration over time.

The second surface of the GTR/GBR scaffold supports gingiva epithelial cell attachment and proliferation, but limits penetration. Over time, degradation of the GTR/GBR scaffold permits integration of the regenerated alveolar bone to the gingiva tissue (FIG. 3 and FIG. 9).

### Kits of the Invention

The invention also includes a kit comprising components useful within the methods of the invention and instructional material that describes, for instance, the method of using the biomimetic scaffolds as described elsewhere herein. The kit may comprise components and materials useful for performing the methods of the invention. For instance, the kit may comprise polymers, solvents, and antisolvents. In certain embodiments, the kit may comprise preformed biomimetic scaffolds. In other embodiments, the kit further comprises cell cultures and surgical instruments.

In one embodiment, the kit is for regenerative endodontics. For example, the kit may comprise biomimetic scaffolds for endodontic regeneration, as described elsewhere herein. In various embodiments, the kit may comprise biomimetic scaffolds having preset sizes, such as small, medium, large, and extra-large, wherein an operator may select an appropriate kit having an appropriately sized scaffold to fit in a root canal in need of endodontic regeneration. The kit may further comprise growth factors or other drugs to enhance endodontic regeneration.

In some embodiments, the kit may further comprise biomimetic scaffolds placed in a preservative from about 0.005% to 2.0% by total weight of the composition. The preservative is used to prevent spoilage in the case of exposure to contaminants in the environment. Examples of preservatives useful in accordance with the invention included but are not limited to those selected from the group consisting of benzyl alcohol, sorbic acid, parabens, imidurea, and combinations thereof. In one embodiment, the preservative is a combination of about 0.5% to 2.0% benzyl alcohol and 0.05% to 0.5% sorbic acid.

In certain embodiments, the kit comprises instructional material. Instructional material may include a publication, a recording, a diagram, or any other medium of expression which can be used to communicate the usefulness of the device or implant kit described herein. The instructional material of the kit of the invention may, for example, be affixed to a package which contains one or more instruments which may be necessary for the desired procedure. Alternatively, the instructional material may be shipped separately from the package, or may be accessible electronically via a communications network, such as the Internet.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1

### Tailoring Porous Degradable Biomaterials for Guided Tissue Regeneration in Regenerative Endodontics

Regenerative Endodontics (RE) is a biologically based procedure to create a new pulp-dentin complex (PDC) in the root canal system (FIG. 13). RE applies principles of regenerative medicine and tissue engineering, utilizing a combination of stem cells, 3-D scaffolds, and growth factors. The spatial control of regenerating tissue is currently a major challenge in RE. The purpose of this study was to develop a biodegradable directional porous scaffold that can address this challenge. This feature supports directional cell penetration and helps regain cellular organization as seen in native PDC.

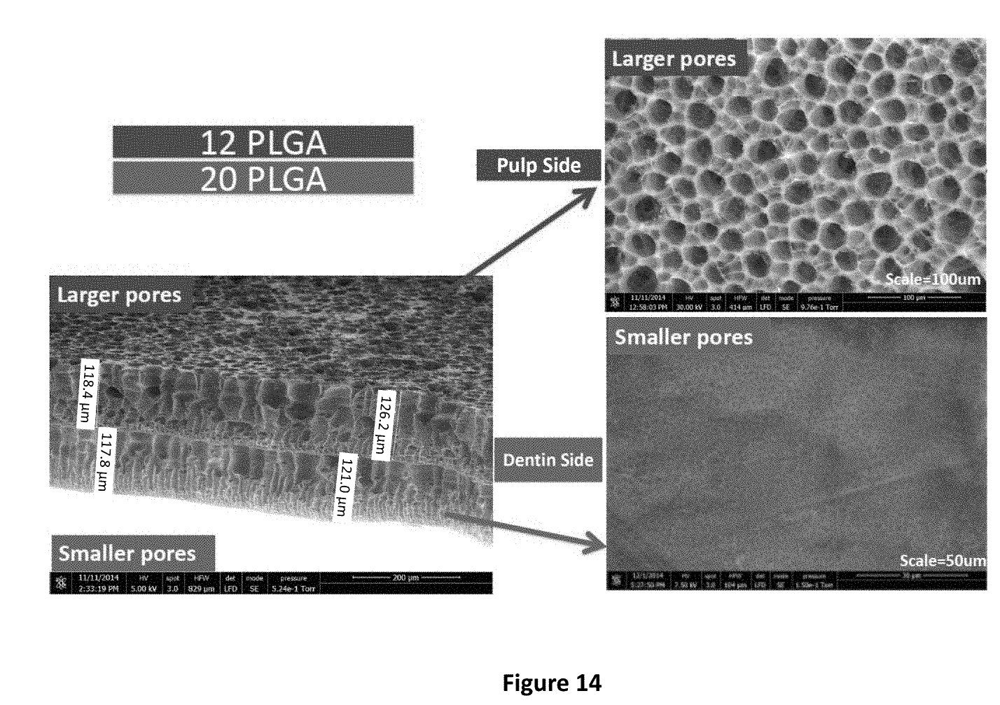

Solvent casting processes were used to develop a poly (lactic-co-glycolic) acid (PLGA) based scaffold. PLGA was solvated in dimethyl sulfoxide (DMSO) at 12% and 20% (w/v) to create two scaffolds having two distinct pore morphologies. A third scaffold was created by laminating the two aforementioned scaffolds together with DMSO. Pore morphology was assessed via scanning electron microscope and confocal microscopy (FIG. 14). Cytocompatibility was evaluated by seeding dental pulp stem cells (DPSC) for 14 days onto the scaffolds. Laser scanning confocal microscopy was used to analyze cell survival, proliferation, and penetration.

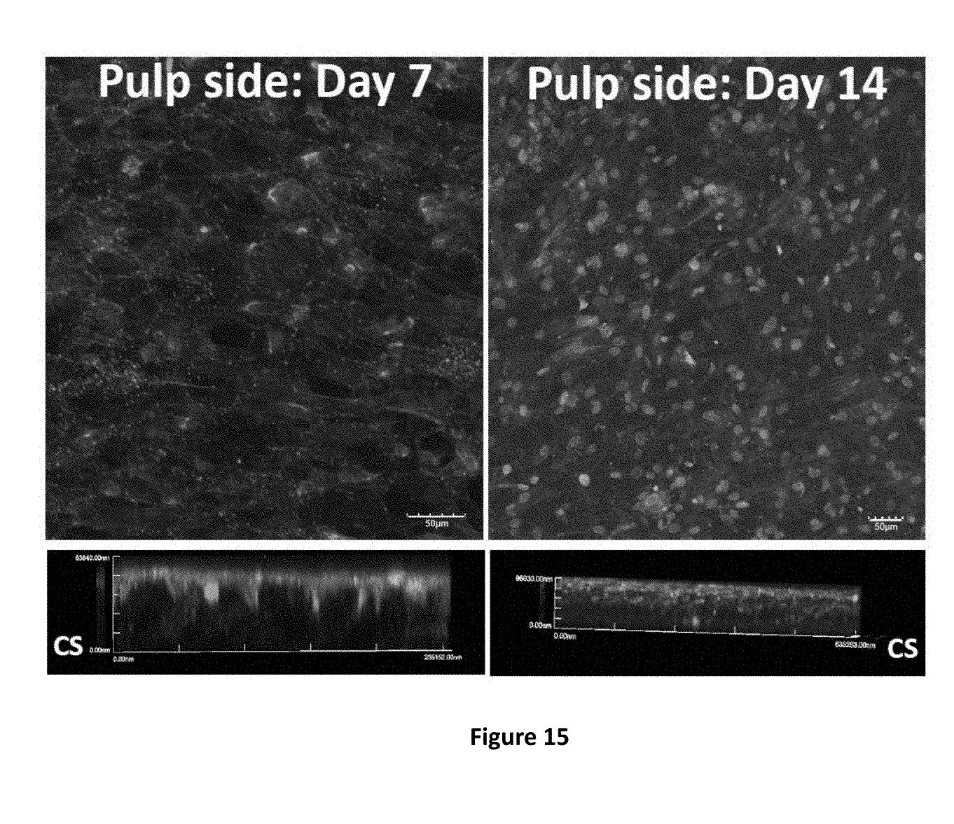

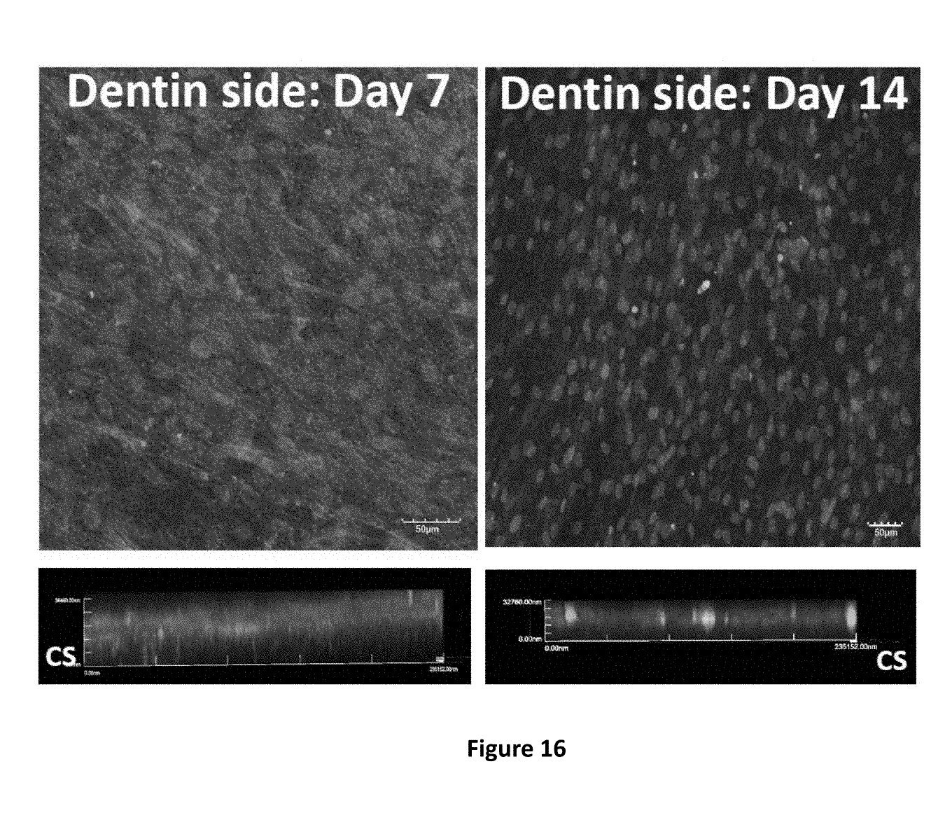

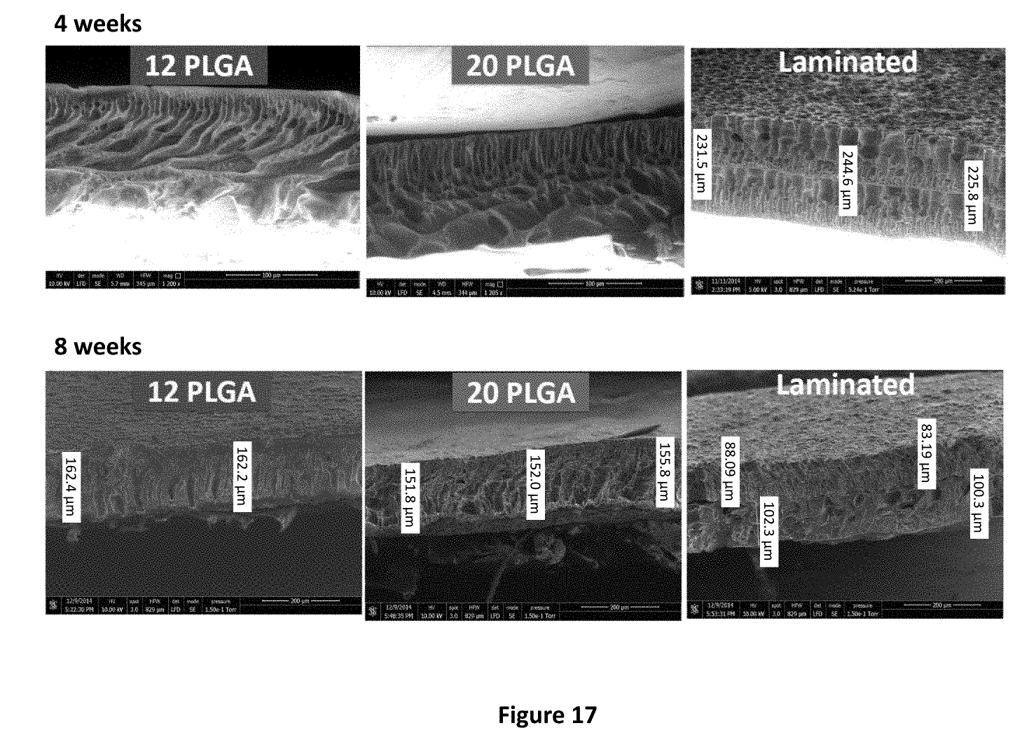

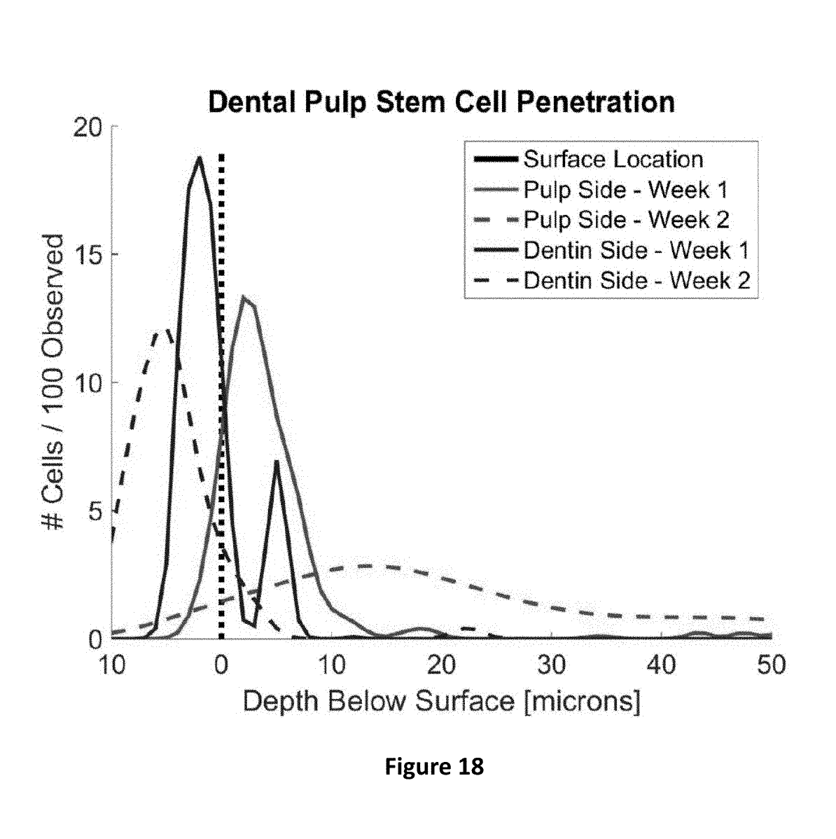

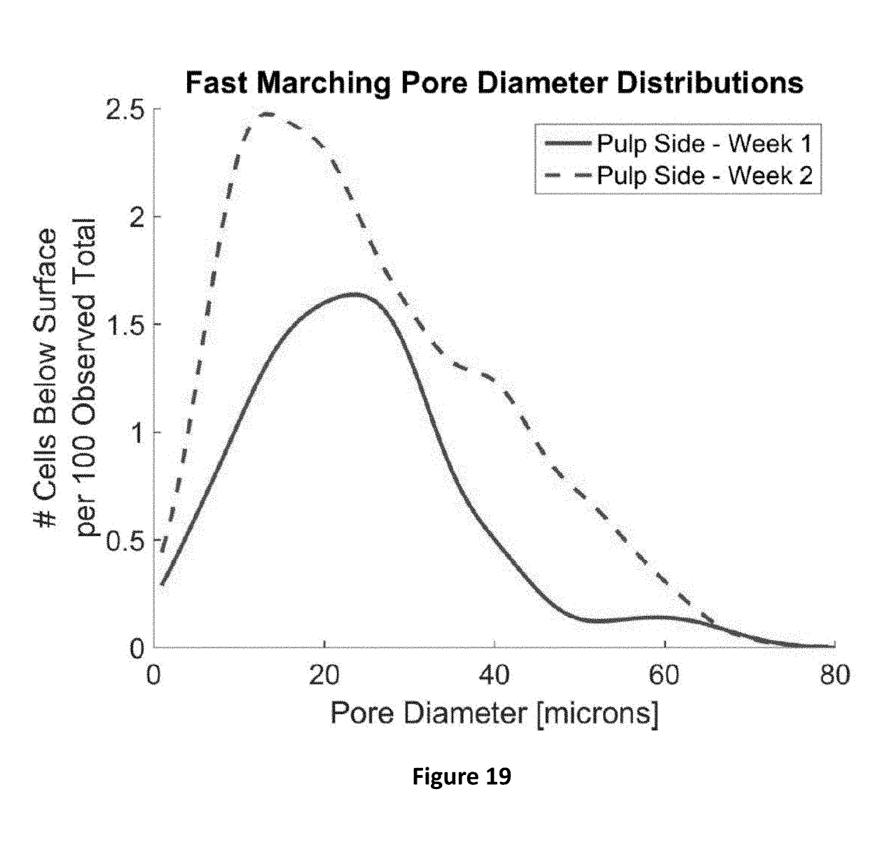

Scaffold thicknesses were 115±30 μm for 12% PLGA, 169±8 μm for 20% PLGA, and 277±15 μm for the combination. The scaffolds had continuous microchannels that reduced in diameter from one side to the other, from 80 to 10 μm and from 10 to 5 μm, for the individual 12% and 20% scaffolds respectively. DPSC survived and proliferated on all the scaffold surfaces and penetrated through the entire thickness of the 12% side of the scaffold in 14 days (FIG. 15, FIG. 18, and FIG. 19). The 20% scaffold predominantly showed cell proliferation on the surface with minimum penetration (FIG. 16, FIG. 18, and FIG. 19). Furthermore, the 12% PLGA scaffold showed more degradation over time than the 20% PLGA scaffold (FIG. 17).

The 12% PLGA scaffold is suitable for regenerating dental pulp tissue because it allows cells to penetrate further into the scaffold. The 20% PLGA scaffold is suitable for regenerating the dentin layer because it allows cells to grow in multiple layers on its surface without significant penetration. The laminated scaffold can be used as an adjunct to current RE techniques by providing spatial guidance to cells on the pulp side for their migration towards the dentin side, by preventing early cell invasion from the dentin side, by maintaining space for pulp regeneration, and by separating cells that have received ECM cues on the dentin side and those on the pulp side.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A biomimetic scaffold material comprising:
a material having a first surface and a second surface; and
a plurality of microchannels extending through the material from the first surface to the second surface;
wherein the diameter of the microchannels is tapered, such that the openings to the microchannels from the first surface have a diameter that is greater than the diameter of the openings to the microchannels from the second surface.

2. A multi-layered biomimetic scaffold material comprising:
at least two layers in contact with each other, each layer comprising a material having a first surface and a second surface; and
a plurality of microchannels extending from the first surface to the second surface;
wherein the diameter of the microchannels is tapered, such that the openings to the microchannels from the first surface have a diameter that is greater than the diameter of the openings to the microchannels from the second surface.

3. The scaffold material of claim 1, wherein the material has a thickness between 10 μm and 10 mm.

4. The scaffold material of claim 1, wherein the microchannel openings from the first surface have a diameter between 10 and 1000 μm.

5. The scaffold material of claim 1, wherein the microchannel openings from the second surface have a diameter between 1 and 10 μm.

6. The scaffold material of claim 1, wherein at least two scaffold materials are combined end-to-end.

7. The scaffold material of claim 1, wherein the material is a biodegradable material.

8. The scaffold material of claim 2, wherein the first layer has a degradation rate that is faster than the at least second layer.

9. The scaffold material of claim 1, wherein the material comprises poly(lactic-co-glycolic acid) (PLGA).

10. The scaffold material of claim 1, wherein the scaffold material is cylindrical.

11. A method of making a biomimetic scaffold, comprising the steps of:
mixing a polymer with a solvent to create a polymer and solvent mixture;
casting the polymer and solvent mixture on a glass plate; and
submerging the glass plate in an antisolvent bath.

12. A method of making a multi-layered biomimetic scaffold, comprising the steps of:
making a first layer using the method of claim 11;
making at least one second layer using the method of claim 11; and
securing the at least two layers together.

13. The method of claim 11, wherein the antisolvent bath is changed at set intervals.

14. The method of claim 11, further comprising a step of lyophilizing the scaffold to remove excess moisture.

15. The method of claim 11, wherein the polymer is PLGA.

16. The method of claim 11, wherein the solvent is dimethyl sulfoxide (DMSO).

17. The method of claim 11, wherein the antisolvent is water.

18. A method of periodontal ligament regeneration, the method comprising the steps of:
providing a biomimetic scaffold comprising a first layer and a second layer; and
inserting the biomimetic scaffold into the space between a tooth and alveolar bone such that the first layer contacts the cementum of the tooth and the second layer contacts the alveolar bone.

19. The method of claim 18, wherein the first layer of the biomimetic scaffold contacts the cementum with a first surface comprising microchannels having first openings of 50-80 μm and contacts the second layer with a second surface comprising microchannels having second openings of 5-10 μm.

20. The method of claim 18, wherein the second layer of the biomimetic scaffold contacts the first layer with a first surface comprising microchannels having first openings of 20-30 μm and contacts the alveolar bone with a second surface comprising microchannels having second openings of less than 5 μm.

21. A method of endodontic regeneration, the method comprising the steps of:
providing a biomimetic scaffold having a first layer and a second layer; and
inserting the biomimetic scaffold into the root canal space such that the first layer faces the interior of the root canal space and the second layer contacts the dentin.

22. The method of claim 21, wherein the first layer of the biomimetic scaffold faces the interior of the root canal space with a first surface comprising microchannels having first openings of 50-80 μm and contacts the second layer with a second surface comprising microchannels having second openings of 5-10 μm.

23. The method of claim 21, wherein the second layer of the biomimetic scaffold contacts the first layer with a first surface comprising microchannels having first openings of 20-30 μm and contacts the dentin with a second surface comprising microchannels having second openings of less than 5 μm.

24. A method of guided tissue regeneration/guided bone regeneration, the method comprising the steps of:
providing a biomimetic scaffold having a first surface and a second surface; and
inserting the biomimetic scaffold into the alveolar bone defect space such that the first surface faces the bony defect and the second surface contacts the gingiva.

25. The method of claim 24, wherein the first surface of the biomimetic scaffold comprises microchannels having first openings of 20-30 μm.

26. The method of claim 24, wherein the second surface of the biomimetic scaffold comprises microchannels having second openings of 5-10 μm.

27. A kit for repairing tissue, comprising at least one biomimetic scaffold material of claim 1.

28. The kit of claim 27, further comprising instructional material for performing the methods of claim 18.

29. The kit of claim 27, wherein the at least one biomimetic scaffold material is provided in a preset size.
